(19) United States
(12) Patent Application Publication
(10) Pub. No.: US 2020/0108043 A1
(43) Pub. Date: Apr. 9, 2020

(54) COMPOUNDS AND COMPOSITIONS FOR EXTENDING LIFESPAN OF A SUBJECT

(71) Applicant: YALE UNIVERSITY, New Haven, CT (US)

(72) Inventors: Murat Acar, New Haven, CT (US); Ethan Sarnosky, Hebron, CT (US)

(21) Appl. No.: 16/624,195

(22) PCT Filed: Jun. 18, 2018
(86) PCT No.: PCT/US2018/038069

§ 371 Date: Dec. 18, 2019

Related U.S. Application Data
(60) Provisional application No. 62/522,764, filed on Jun. 21, 2017.

(51) Int. Cl. A61K 31/336; A61K 31/4453; A61K 31/443; A61K 31/196; A61K 31/473; A61K 31/7056; A61K 31/52; A61K 31/343; A61K 31/417; A61K 31/5415; A61K 31/155; A61K 31/352; A61K 31/42; A61K 33/24; A61K 31/436; A61K 31/192; A61K 31/7076

(52) U.S. Cl. CPC A61K 31/336; A61K 31/7076; A61K 31/443; A61K 31/196; A61K 31/473; A61K 31/7056; A61K 31/52; A61K 31/343; A61K 31/417; A61K 31/5415; A61K 31/155; A61K 31/352; A61K 31/42; A61K 33/24; A61K 31/436; A61K 31/192; A61K 31/4453

(57) ABSTRACT

The present invention relates in part to the unexpected discovery that certain compounds extend the lifespan of eukaryotic organisms. In certain embodiments, the invention comprises a method of extending the lifespan of a subject comprising administering to the subject a therapeutically effective amount of at least one compound selected from the group consisting of terreic acid and mycophenolic acid. The invention further relates to methods for screening potential compounds of interest for lifespan extending properties.

### CROSS-REFERENCE TO RELATED APPLICATIONS

This application claims priority under 35 U.S.C. § 119(e) to U.S. Provisional Application No. 62/522,764, filed Jun. 21, 2017, all of which is incorporated herein by reference in its entirety.

### STATEMENT REGARDING FEDERALLY SPONSORED RESEARCH OR DEVELOPMENT

This invention was made with government support under AG050461 awarded by National Institutes of Health and under 1122492 awarded by the National Science Foundation. The government has certain rights in the invention.

### BACKGROUND OF THE INVENTION

Aging is the greatest risk factor for morbidity and mortality throughout the developed world. Thus, one could in principle extend healthy lifespan by modulating the aging process. However, the few such interventions described so far, including mTOR inhibition and dietary restriction, have not been met with wide success. While existing human therapeutics have great potential to improve health in old age, further research is needed to eliminate age-related diseases themselves.

One of the greatest impediments to the progress of aging research is the fundamental time-requirement of longitudinal aging studies. The lifespan of model organisms can range from years in mammals to several days in the yeast Saccharomyces cerevisiae. Throughput limitations have been partially addressed through massive parallel studies in the moderately long-lived organism Caenorhabditis elegans, or technology that enables rapid, but not scalable, experiments in short-lived models. However, these approaches are constrained in that they permit either large-scale or quick turn-around, but not both.

There remains a need in the art for compounds and compositions that can be used to extend healthy lifespan in a subject. There also remains a need for methods of testing and screening for compositions and methods capable of extending the lifespan of a subject. The present invention addresses these needs.

### BRIEF SUMMARY OF THE INVENTION

In one aspect, the invention provides a method of extending the lifespan of a subject. In certain embodiments, the method comprises administering to the subject a therapeutically effective amount of at least one compound, or a salt, solvate, enantiomer, diastereoisomer, or tautomer thereof, selected from the group consisting of:

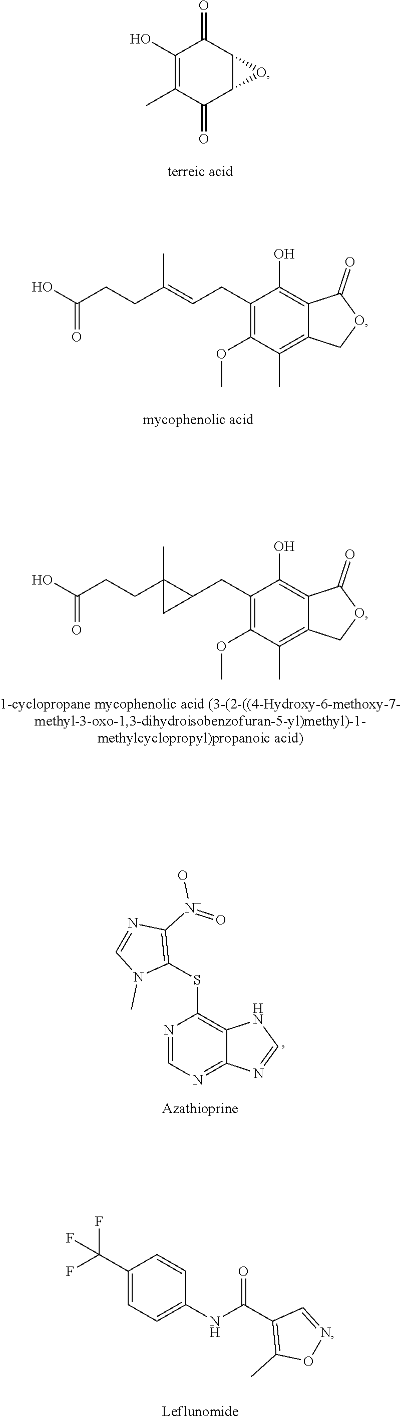

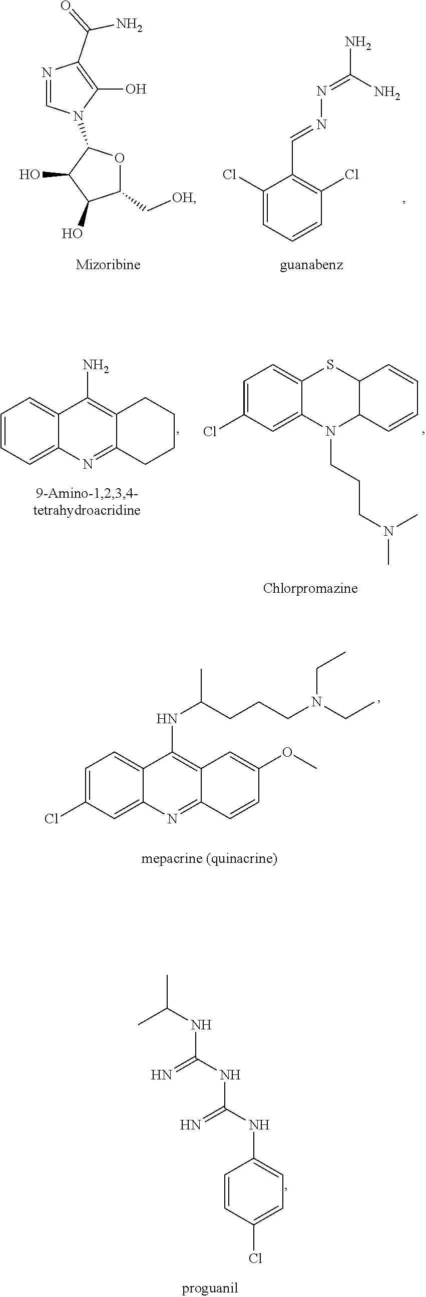

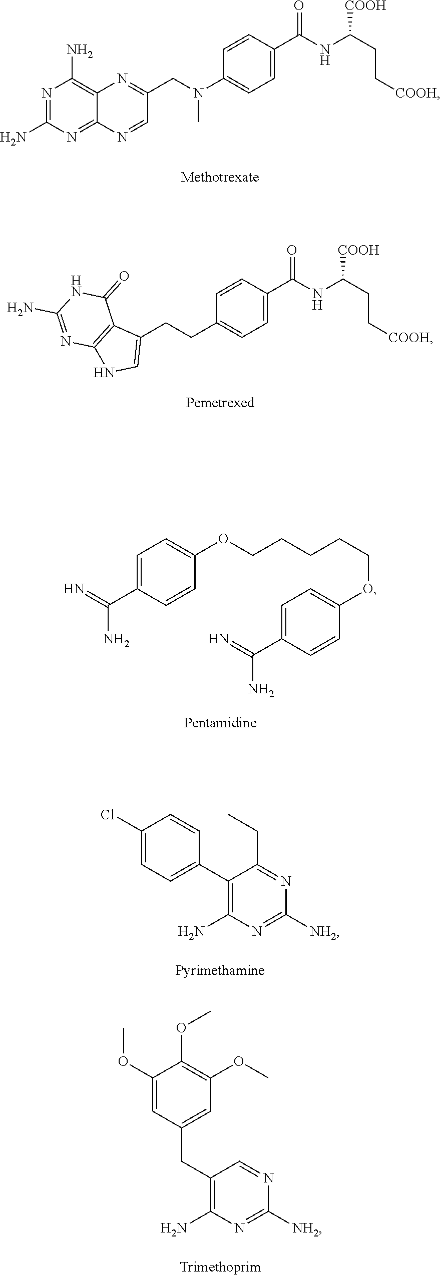 [SMILES: CN(Cc1cnc2nc(N)nc(N)c2n1)c1ccc(C(=O)N[C@@H](CCC(=O)O)C(=O)O)cc1 ; Nc1nc2[nH]cc(CCc3ccc(C(=O)N[C@@H](CCC(=O)O)C(=O)O)cc3)c2c(=O)[nH]1 ; N=C(N)c1ccc(OCCCCCOc2ccc(C(=N)N)cc2)cc1 ; CCc1nc(N)nc(N)c1-c1ccc(Cl)cc1 ; COc1cc(Cc2cnc(N)nc2N)cc(OC)c1OC]

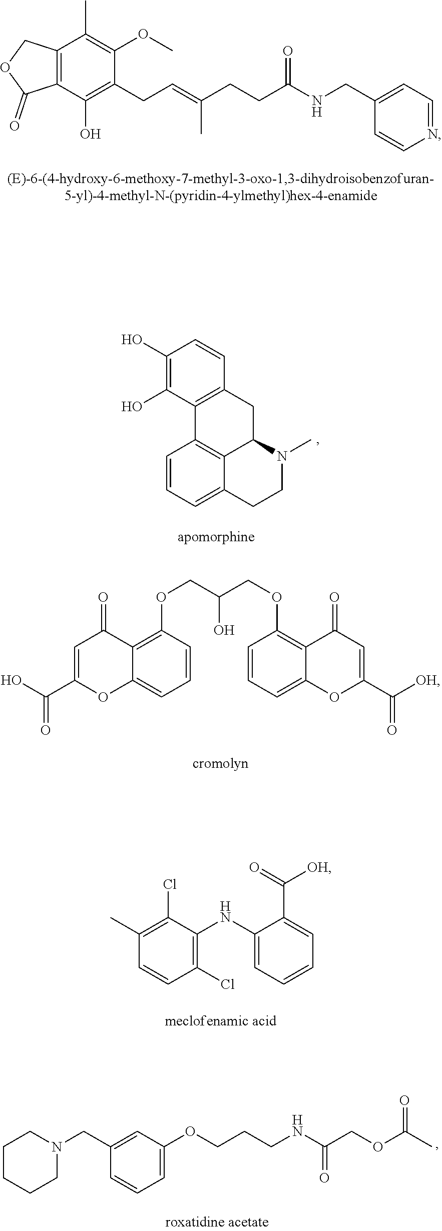 [SMILES: COc1c(C)c2c(c(O)c1C/C=C(\C)CCC(=O)NCc1ccncc1)C(=O)OC2 ; CN1CCc2cccc3c2[C@H]1Cc1ccc(O)c(O)c1-3 ; O=C(O)c1cc(=O)c2c(OCC(O)COc3cccc4oc(C(=O)O)cc(=O)c34)cccc2o1 ; Cc1ccc(Cl)c(Nc2ccccc2C(=O)O)c1Cl ; CC(=O)OCC(=O)NCCCOc1cccc(CN2CCCCC2)c1]

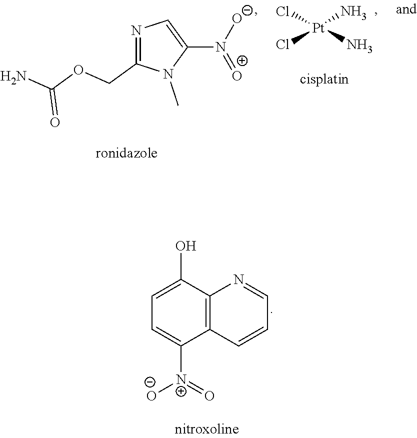

In certain embodiments, the lifespan of the subject is extended by about 15% to about 25%. In other embodiments, wherein the lifespan of the subject is extended by about 18% to about 23%.

In certain embodiments, the at least one compound treats an aging-related disease or disorder. In other embodiments, the aging-related disease or disorder is one or more selected from the group consisting of atherosclerosis, cardiovascular disease, respiratory disease, cancer, arthritis, osteoporosis, type 2 diabetes, hypertension, Alzheimer's disease, Parkinson's disease, liver disease, kidney disease, and immunosenescence.

In certain embodiments, the at least one compound alters immune response in the subject. In other embodiments, the at least one compound suppresses the subject's immune system.

In certain embodiments, the at least one compound inhibits guanosine monophosphate (GMP) synthesis in the subject. In other embodiments, the at least one compound inhibits the synthesis of tetrahydrofolate in the subject.

In certain embodiments, the at least one compound is administered as part of a pharmaceutical composition. In other embodiments, the subject is further administered at least one additional agent useful for extending lifespan. In yet other embodiments, the at least one compound and the at least one additional agent are co-formulated. In yet other embodiments, the at least one additional agent useful for extending lifespan is selected from the group consisting of ibuprofen, rapamycin, metformin, and nicotinamide riboside.

In certain embodiments, the subject is a eukaryotic organism. In other embodiments, the subject is a mammal. In yet other embodiments, the subject is a human.

In another aspect, the invention provides a method of identifying compounds that extend the lifespan of a subject. In certain embodiments, the method comprises contacting “mother enriched” yeast cells with an NHS functionalized fluorophore in a growth medium, to form a first system. In other embodiments, the method comprises contacting at least one aliquot of the first system with β-estradiol, to form a second system. In yet other embodiments, the method comprises incubating the second system with a test compound or control compound, to form a third system. In yet other embodiments, the method comprises contacting the third system with a WGA functionalized fluorophore and a cell viability dye, to form a fourth system. In other embodiments, the method comprises conducting flow cytometry on the fourth system to detect fluorescence from at least one fluorophore selected [symbol] from the group consisting of the NHS functionalized fluorophore, the WGA functionalized fluorophore and the cell viability dye. In yet other embodiments, the “mother enriched” yeast cells are genetically modified yeast cells wherein the replicative capacity of the “mother enriched” yeast cells is not altered while the replicative capacity of their progeny cells is restricted.

In certain embodiments, the NHS functionalized fluorophore is at least one selected from the group consisting of NHS-Fluorescein and NHS-Rhodamine.

In certain embodiments, the cell viability dye is propidium iodide.

In certain embodiments, the WGA functionalized fluorophore is CF405M-WGA.

In certain embodiments, the mean lifespan of the yeast cells is determined by conducting flow cytometry on each sample at two or more time points. In other embodiments, flow cytometry is conducted at two or more time points between 0 hours and about 48 hours.

In certain embodiments, the flow cytometry is carried out using an automated flow cytometry device.

In certain embodiments, the at least one aliquot is part of a screening array. In other embodiments, the screening array comprises a multi-well plate.

### BRIEF DESCRIPTION OF THE DRAWINGS

The following detailed description of specific embodiments of the invention will be better understood when read in conjunction with the appended drawings. For the purpose of illustrating the invention, there are shown in the drawings specific embodiments. It should be understood, however, that the invention is not limited to the precise arrangements and instrumentalities of the embodiments shown in the drawings.

FIG. 1 is a work-flow diagram of certain screening methods of the invention. The progenitor cell population of interest is persistently labeled with an NETS-Ester fluorescein conjugate, which asymmetrically segregates to the mother cell during division. The fraction of cells viable within the progenitor population is then determined using propidium iodide live cells exclude the red dye. Finally, the replicative age of viable progenitor cells is measured using wheat germ agglutinin conjugated to a blue fluorophore, which labels bud scars left behind with each division. A complete lifespan curve can be constructed using serial measurements taken over the course of 2-3 days.

FIGS. 2A-2G are schemes and graphs showing validation of the High-Life (High throughput replicative Lifespan measurement) system of the invention. FIG. 2A is a [symbol] schematic representation of the Mother Enrichment Program (MEP). β-estradiol inducible Cre-recombinase is expressed under the control of a daughter cell specific promoter, P_{SCW11}. LoxP sites are integrated into surrounding components of the essential genes ubc9 and cdc20. FIG. 2B is a graph showing the fold-change in MEP cells inoculated at different initial densities plotted over time. Error bars are S.E.M. for 6 independent replicates. FIG. 2C is a graph of the fraction of viable cells plotted against replicative age for fluorescein-labeled versus unlabeled cells. N=100 cells for each group; mean lifespans were 22.9 and 22.4, respectively. FIG. 2D is a graph showing the total number of cells that fall into the fluorescein-positive fraction at various times during a representative High-Life experiment. FIG. 2E is a graph showing the fraction of all cells that fall into the fluorescein-positive fraction at various times during a representative High-Life experiment. FIG. 2F is a graph showing the fraction of viable progenitor cells plotted against time, beginning after either birth of the cell (Replicator) or initiation of the culture (High-Life), for representative experiments. A third line (False-Positive Adjusted) represents that fraction of viable cells in the High-Life environment after correcting for the false-positive rate observed with propidium iodide staining in the Replicator device. Correction was performed by extrapolating a linear trend of false-positives between cells in the Replicator device stained with propidium iodide after 16 or 40 hours of culture, and multiplying the observed High-Life viability by 1 minus the calculated false-positive fraction. 100 cells were considered for the Replicator experiment. The High-Life experiment shows the mean of 48 replicate wells. FIG. 2G is a graph showing the mean number of bud scars observed on fluorescein-labeled cells after 0, 8, and 24 hours of culture, plotted against the CF405M fluorescence intensity observed at the same timepoint. For bud scar counting, 20 cells were analyzed at each timepoint. CF405M intensity values are the mean of 48 replicate wells.

FIGS. 3A-3G are a set of graphs reporting detection of lifespan extension using High-Life. FIG. 3A is a graph of the fraction of progenitor cells viable plotted against the corresponding blue fluorescence intensity, with timepoints taken at 0, 8, 16, 24, 28, 32, 36, 40, and 48 hours after labeling. Two separate experiments are shown, each comparing ibuprofen treated samples to an untreated control. Error bars are S.E.M. of 48 replicates.

FIG. 3B is a plot of the individual replicate points that compose the mean values shown in FIG. 3A. The solid line is the result of second order polynomial fitting on pooled data from both untreated experiments; the dashed lines denote a 95% confidence interval. FIG. 3C is a graph of the fraction of wells in the ibuprofen condition that fall below the upper boundary of the 95% confidence interval (false negatives), and fraction of untreated wells that fall above [symbol] the upper boundary of the 95% confidence interval (false positives) at each timepoint sampled. FIGS. 3D-3F are graphs of the fraction of viable progenitor cells plotted against the corresponding blue fluorescence intensity for wild-type, Δfob1 (FIG. 3D), Δgpa2 (FIG. 3E), and Δsgf73 (FIG. 3F) strains sampled at various times after labeling. Error bars are S.E.M. of 12 independent replicates. FIG. 3G is a graph showing the areas between the curve for High-Life experiments performed under the same conditions (left), or with strains expected to exhibit lifespan differences (right). For consistent comparison, areas between the respective curves were computed with Mean CF405M Intensity ranging from 70 to 1100.

FIGS. 4A-4E are graphs of plate based screening using High-Life. FIG. 4A is a graph of progenitor fraction viable plotted against CF405M intensity for individual wells of a 384 well plate based screen. Shown for a single plate are all untreated (DMSO) negative control samples, all ibuprofen-treated positive control samples, and all compounds from this plate that were selected for follow-up. FIG. 4B is a graph of progenitor fraction viable plotted against CF405M intensity for the averages of all wells for control, ibuprofen, and confirmed hit compounds. Rapamycin is separated for demonstration purposes. Error bars are not shown, as S.E.M. bars were generally smaller than the points themselves. FIG. 4C is a graph of dose-response for mycophenolic acid, chosen as a representative of the three compounds with clear concentration-dependent effects selected for follow-up validation. Error bars are S.E.M. for four or more replicate wells. FIGS. 4D-4E are graphs showing dose-response for cells treated with terreic acid (FIG. 4D) or 8-hydroxy-5-nitroquinoline (FIG. 4E) during 24 hours of culture. Error bars are S.E.M. for four or more replicate wells.

FIG. 5 is a graph reporting secondary validation of screening hits. Survival curves and lifespan characteristics for wild-type, haploid cells grown in the absence (untreated) or presence of DMSO vehicle-control, 10 μM terreic acid, or 10 μM mycophenolic acid. In each experiment, lifespan measurements were made on a single-cell level for 100 cells in each condition using a novel microfluidic Replicator device. Each curve contains pooled data from two independent experiments.

FIGS. 6A-6B show that inhibition of GMP synthesis extends yeast replicative lifespans (RLS). FIG. 6A is a simplified schematic representation of GMP synthesis pathways in S. cerevisiae. Mycophenolic acid (MPA) limits de novo GMP synthesis via inhibition of IMD genes. GMP is synthesized via the salvage pathway in the presence of exogenous guanine. FIG. 6B is a set of lifespan curves for wild-type, haploid yeast (BY4741) in the presence or absence of MPA and guanine. N=200 cells for each condition, pooled from two independent experiments of 100 cells each.

FIGS. 7A-7I show that inhibition of GMP synthesis extends lifespan independent of the nutrient sensing and sirtuin pathways. FIG. 7A. is a schematic representation of the LPT test and its interpretation. FIGS. 7B-7C are schamtics showing possible outcomes of the longevity placement test (LPT). Possible network architectures and outcomes of the LPT are shown in step 1 (FIG. 7B) and step 2 (FIG. 7C). Interactions shown in dashed lines represent those that are prevented, either by the suppression agent, or via deletion of the gene. FIGS. 7D-7I are lifespan curves corresponding to Step 1 (FIGS. 7D, 7F and 7H) or Step 2 (FIGS. 7E, 7G and 7I) of the LPT test for the nutrient sensing pathway, including a dietary restriction mimetic (FIGS. 7D-7E) and TOR inhibition (FIGS. 7F-7G), and the sirtuin pathway (FIGS. 7H and 7I). N=200 cells for each condition, pooled from two or more independent experiments.

FIGS. 8A-8F show proteasome activation extends lifespan through GMP depletion. FIGS. 8A-8B are lifespan curves corresponding to Step 1 (FIG. 8A) and Step 2 (FIG. 8B) of the LPT test for the proteasome pathway of lifespan extension. N=200 cells for each condition, pooled from two independent experiments of 100 cells each. FIG. 8C is a graph showing proteasome activity for wild-type (BY4741) cells, or AUBR2 cells, in the presence or absence of MPA or guanine. N=3 biological replicates for each condition. Errors bars are standard error of the mean. NSD, no significant difference. FIG. 8D is a graph showing the negative control for the proteasome activity experiment shown in FIG. 8C. MG-132, a proteasome inhibitor, was added to separate wells of the experiment run concurrently. The low rate of fluorescence increase in the presence of MG-132 indicated that the measurements were specific to the proteasome. FIG. 8E is a lifespan curve for a APRE9 strain in the presence or absence of MPA. N=200 cells for each condition, pooled from two independent experiments of 100 cells each. FIG. 8F is a schematic diagram presenting the relationship of longevity interactions discovered as an aspect of the invention. The actions of MPA converge on the actions of the proteasome at the level of GMP or its downstream metabolites.

FIG. 9A-9C are graphs showing that MPA slows accumulation of age-related damage in yeast. FIGS. 9A-9C are replicative lifespan curves for S. cerevisiae treated with 10 μM mycophenolic acid (MPA) only during the first 24 hours of a Replicator experiment (FIG. 9A), only after the first 24 hours of a Replicator experiment (FIG. 9B), or between the 24th and 30th hour of a Replicator experiment (FIG. 9C). N=200 cells for each condition, pooled from two independent experiments of 100 cells each.

FIG. 10 is a graph and table showing lifespan extension of S. cerevisiae treated with 10 μM proguanil hydrochloride or 10 μM of guanabenz acetate, as compared to untreated [symbol] control.

FIG. 11 is a graph showing that terreic acid and mycophenolic acid demonstrate lifespan extending properties in an evolutionarily conserved manner. Roundworms (C. elegans) were treated with terreic acid or mycophenolic acid for the duration of their lifespans. In each experiment, statistically significant lifespan extension was observed as compared to untreated control roundworms.

FIG. 12 is a graph showing the results of High-Life tests assessing extension of replicative lifespan in S. cerevisiae for compounds sharing structural similarities with mycophenolic acid. (E)-6-(4-hydroxy-6-methoxy-7-methyl-3-oxo-1,3-dihydroisobenzofuran-5-yl)-4-methyl-N-(pyridin-4-ylmethyl)hex-4-enamide and 3-(2-((4-Hydroxy-6-methoxy-7-methyl-3-oxo-1,3-dihydroisobenzofuran-5-yl)methyl)-1-methylcyclopropyl)propanoic acid demonstrated measurable lifespan extension in initial tests.

FIG. 13 is a graph showing lifespan extension of S. cerevisiae treated with 10 μM proguanil in the presence or absence of 10 ug/mL folinic acid.

### DETAILED DESCRIPTION OF THE INVENTION

The present invention relates in part to the unexpected discovery that certain compounds extend the lifespan of eukaryotic organisms. In certain embodiments, the invention provides a method of extending the lifespan of a subject, the method comprising administering to the subject a therapeutically effective amount of at least one compound selected from the group consisting of terreic acid, mycophenolic acid, guanabenz, proguanil (or chloroguanide), apomorphine, cromolyn, meclofenamic acid, roxatidine acetate, ronidazole, cisplatin, nitroxoline, chlorpromazine, quinacrine, azathioprine, leflunomide, mizoribine, methotrexate, pemetrexed, pentamidine, pyrimethamine, trimethoprim, (E)-6-(4-hydroxy-6-methoxy-7-methyl-3-oxo-1,3-dihydroisobenzofuran-5-yl)-4-methyl-N-(pyridin-4-ylmethyl)hex-4-enamide and 3-(2-((4-Hydroxy-6-methoxy-7-methyl-3-oxo-1,3-dihydroisobenzofuran-5-yl)methyl)-1-methylcyclopropyl)propanoic acid. The invention also relates to methods for efficiently screening potential compounds of interest for lifespan extending properties.

### Definitions

As used herein, each of the following terms has the meaning associated with it in this section.

Unless defined otherwise, all technical and scientific terms used herein have the same [symbol] commonly understood by one of ordinary skill in the art to which this invention belongs. Although any methods and materials similar or equivalent to those described herein can be used in the practice or testing of the present invention, exemplary methods and materials are described.

Generally, the nomenclature used herein and the laboratory procedures in organic chemistry and cell culturing are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

As used herein, the term “ED₅₀” or “ED50” refers to the effective dose of a formulation that produces about 50% of the maximal effect in subjects that are administered that formulation.

As used herein, an “effective amount,” “therapeutically effective amount” or “pharmaceutically effective amount” of a compound is that amount of compound that is sufficient to provide a beneficial effect to the subject to which the compound is administered.

“Instructional material,” as that term is used herein, includes a publication, a recording, a diagram, or any other medium of expression that can be used to communicate the usefulness of the composition and/or compound of the invention in a kit. The instructional material of the kit may, for example, be affixed to a container that contains the compound and/or composition of the invention or be shipped together with a container that contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

As used herein, a “patient” or “subject” can be a human or non-human mammal or a [symbol] Non-human mammals include, for example, livestock and pets, such as ovine, bovine, porcine, canine, feline and murine mammals. In certain embodiments, the subject is human.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound include, but are not limited to, intravenous, oral, aerosol, parenteral, ophthalmic, pulmonary and topical administration.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that [symbol] ible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, inorganic bases, solvates, hydrates, and clathrates thereof.

The term “prevent,” “preventing” or “prevention,” as used herein, means avoiding or delaying the onset of symptoms associated with a disease or condition in a subject that has not developed such symptoms at the time the administering of an agent or compound commences. Disease, condition and disorder are used interchangeably herein.

The term “solvate,” as used herein, refers to a compound formed by solvation, which is a process of attraction and association of molecules of a solvent with molecules or ions of a solute. As molecules or ions of a solute dissolve in a solvent, they spread out and become surrounded by solvent molecules.

The term “treat,” “treating” or “treatment,” as used herein, means reducing the frequency or severity with which symptoms of a disease or condition are experienced by a subject by virtue of administering an agent or compound to the subject.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following abbreviations are used herein: CF405M-WGA, wheat germ agglutinin [symbol] to CF405M dye; GMP, guanosine monophosphate; GTP, guanosine triphosphate; High-Life, High throughput replicative Lifespan measurement; IMD, inosine monophosphate dehydrogenase; LPT, Longevity Placement Test; MEP, Mother Enrichment Program; MPA, mycophenolic acid; NGM, Nematode Growth Media; NHS, N-hydroxysuccinimide; RLS, Replicative Lifespan; WGA, wheat germ agglutinin.

### Methods of Extending Lifespan

The invention includes methods of extending the lifespan of a eukaryotic subject. In certain embodiments, the method comprises administering to the subject a therapeutically effective amount of at least one compound selected from the group consisting of:

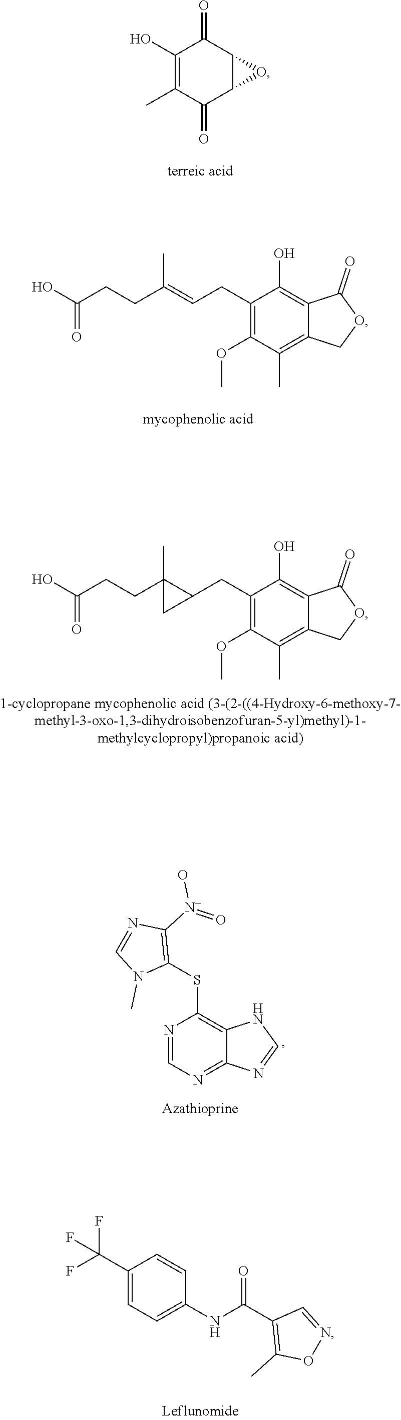

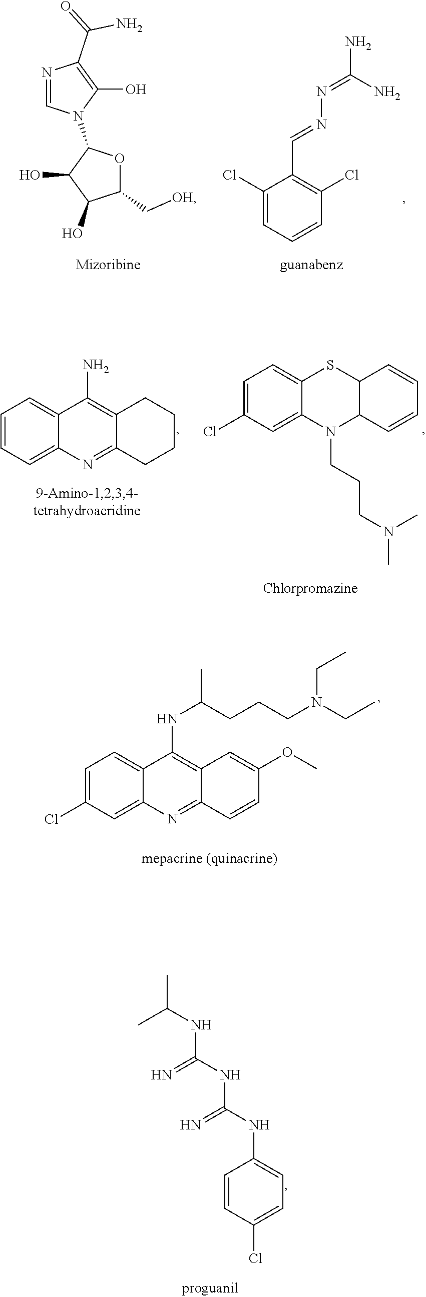

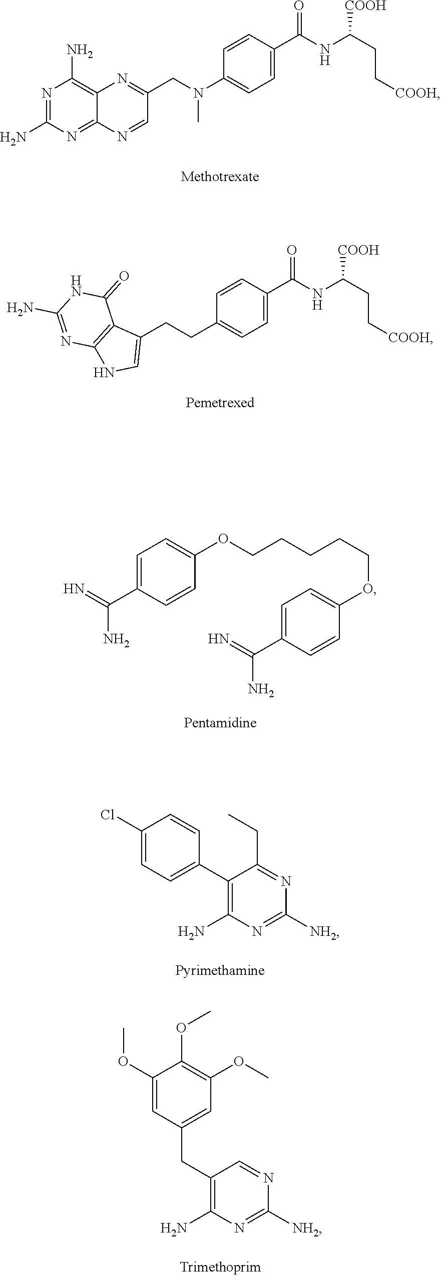 [SMILES: CN(Cc1cnc2nc(N)nc(N)c2n1)c1ccc(C(=O)N[C@@H](CCC(=O)O)C(=O)O)cc1 ; Nc1nc2[nH]cc(CCc3ccc(C(=O)N[C@@H](CCC(=O)O)C(=O)O)cc3)c2c(=O)[nH]1 ; N=C(N)c1ccc(OCCCCCOc2ccc(C(=N)N)cc2)cc1 ; CCc1nc(N)nc(N)c1-c1ccc(Cl)cc1 ; COc1cc(Cc2cnc(N)nc2N)cc(OC)c1OC]

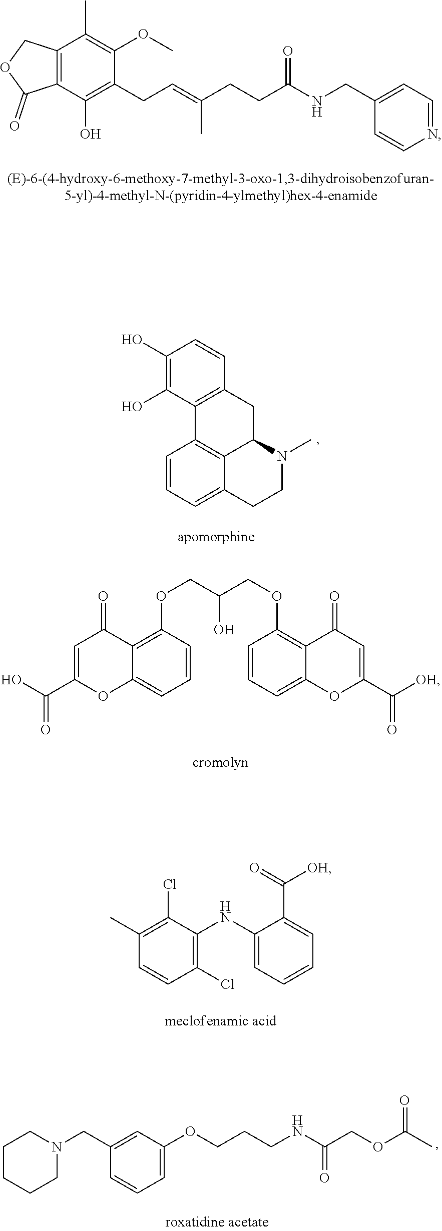 [SMILES: COc1c(C)c2c(c(O)c1C/C=C(\C)CCC(=O)NCc1ccncc1)C(=O)OC2 ; CN1CCc2cccc3c2[C@H]1Cc1ccc(O)c(O)c1-3 ; O=C(O)c1cc(=O)c2c(OCC(O)COc3cccc4oc(C(=O)O)cc(=O)c34)cccc2o1 ; Cc1ccc(Cl)c(Nc2ccccc2C(=O)O)c1Cl ; CC(=O)OCC(=O)NCCCOc1cccc(CN2CCCCC2)c1]

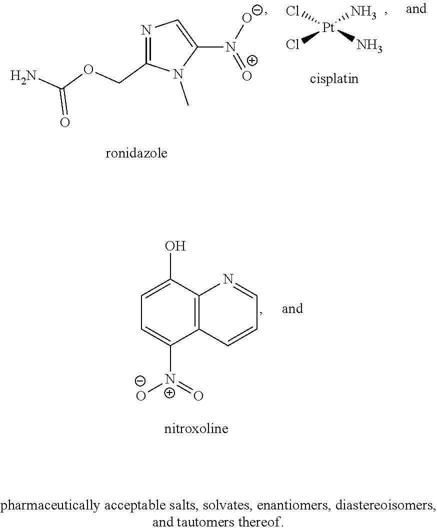

In certain embodiments, the methods of the invention extend the lifespan of the subject by about 15% to about 25% compared to a control. In other embodiments, the methods of the invention extend the lifespan of the subject by about 18% to about 23% compared to a control.

Without being limited to any one theory, in certain embodiments, the methods of the invention extend lifespan of the subject by treating an aging-related disease or disorder. In other embodiments, the aging-related disease or disorder is one or more diseases or disorders selected from the group consisting of atherosclerosis, cardiovascular disease, respiratory disease, cancer, arthritis, osteoporosis, type 2 diabetes, hypertension, Alzheimer's disease, Parkinson's disease, liver disease, kidney disease, or immunosenescence Without being limited to any one theory, in certain embodiments, the methods of the invention extend lifespan of the subject by altering immune response in the subject. In other embodiments, the compounds of the invention suppress the subject's immune system.

Without being limited to any theory, in certain embodiments, the methods of the invention extend lifespan of the subject by inhibiting at least one selected from the group consisting of guanosine monophosphate (GMP) synthesis, adenosine monophosphate (AMP) synthesis, and tetrahydrofolate synthesis

In certain embodiments, the compounds of the invention are administered to a subject in combination with at least one additional compound which are known to increase lifespan in a subject. In other embodiments, the at least one additional compound is administered at the same time as the compounds of the invention. In yet other embodiments, the at least one compound of the invention and the at least one additional compound are co-formulated into a pharmaceutical composition. In certain embodiments, the at least one additional compound is at least one compounds selected from the group consisting of ibuprofen, rapamycin, metformin, and nicotinamide riboside.

In certain embodiments, the methods of the invention comprise the use of the at least one compound to extend lifespan in a prophylactic capacity. The at least one compound is administered at any point during the lifespan of the subject, regardless of the health or disease state of the subject. In other embodiments, the methods of the invention are applied throughout the entire lifetime of the subject. In other embodiments, the methods of the invention are applied late in life (an adult and/or a mature adult), or after the onset of disease. In yet other embodiments, the compounds of the invention are formulated for continuous, indefinite daily use.

In certain embodiments, the subject is a single cell organism. In other embodiments, the subject is a yeast cell. In yet other embodiments, the subject is a mammal. In yet other embodiments, the subject is a human.

The compounds used in the methods described herein may form salts with acids and/or bases, and such salts are included in the present invention. In certain other embodiments, the salts are pharmaceutically acceptable salts. The term “salts” embraces addition salts of free acids and/or bases that are useful within the methods of the invention. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hemisulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric, galacturonic acid, glycerophosphonic acids and saccharin (e.g., saccharinate, saccharate).

Suitable pharmaceutically acceptable base addition salts of compounds used in the methods of the invention include, for example, metallic salts including alkali metal, alkaline earth metal and transition metal salts such as, for example, calcium, magnesium, potassium, [symbol] and zinc salts. Pharmaceutically acceptable base addition salts also include organic salts made from basic amines such as, for example, ammonium, N,N′-dibenzylethylene-diamine, chloroprocaine, choline, diethanolamine, ethylenediamine, meglumine (N-methylglucamine) and procaine.

All of these salts may be prepared from the corresponding compound by reacting, for example, the appropriate acid or base with the compound. Salts may be comprised of a fraction of less than one, one, or more than one molar equivalent of acid or base with respect to any compound of the invention.

In certain other embodiments, the at least one compound of the invention is a component of a pharmaceutical composition further including at least one pharmaceutically acceptable carrier.

The compounds used in the methods of the invention may possess one or more stereocenters, and each stereocenter may exist independently in either the (R) or (S) configuration. In certain other embodiments, compounds described herein are present in optically active or racemic forms. The compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain other embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound of the invention, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain other embodiments, the compounds described herein exist in solvated forms with pharmaceutically acceptable [symbol] such as water, and ethanol. In other embodiments, the compounds described herein exist in unsolvated form.

In certain other embodiments, the compounds of the invention exist as tautomers. All tautomers are included within the scope of the compounds recited herein.

In certain other embodiments, compounds described herein are prepared as prodrugs. A “prodrug” is an agent converted into the parent drug in vivo. In certain other embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain other embodiments, sites on, for example, the aromatic ring portion of compounds of the invention are susceptible to various metabolic reactions. Incorporation of appropriate substituents on the aromatic ring structures may reduce, minimize or eliminate this metabolic pathway. In certain other embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain other embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain other embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and in the art. [symbol] methods for the preparation of compound as described herein are modified by the use of appropriate reagents and conditions, for the introduction of the various moieties found in the formula as provided herein.

### Combination Therapies

In one aspect, the compounds of the invention are useful within the methods of the invention in combination with at least one additional agent useful for extending the lifespan of a subject. These additional agents may comprise compounds or compositions identified herein, or compounds (e.g., commercially available compounds) known to extend lifespan, or treat, prevent, or reduce the symptoms of aging-related diseases.

A synergistic effect may be calculated, for example, using suitable methods such as, for example, the Sigmoid-Eₘₐₓ equation (Holford & Scheiner, 1981, Clin. Pharmacokinet. 6:429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22:27-55). Each equation referred to elsewhere herein may be applied to experimental data to generate a corresponding graph to aid in assessing the effects of the drug combination. The corresponding graphs associated with the equations referred to elsewhere herein are the concentration-effect curve, isobologram curve and combination index curve, respectively.

### Administration/Dosage/Formulations

The regimen of administration may affect what constitutes an effective amount. The therapeutic formulations may be administered to the subject either prior to or after the onset of a disease or disorder contemplated in the invention. Alternatively, the therapeutic formulations may be administered to the subject continuously or preemptively in order to extend lifespan. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated in the invention. An effective amount of the therapeutic compound necessary to achieve a therapeutic effect [symbol] according to factors such as the state of the disease or disorder in the patient; the age, sex, and weight of the patient; and the ability of the therapeutic compound to treat a disease or disorder contemplated in the invention. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. The pharmaceutical compositions useful for practicing the invention may be administered to deliver a dose of from 1 ng/kg/day and 100 mg/kg/day. One of ordinary skill in the art would be able to study the relevant factors and make the determination regarding the effective amount of the therapeutic compound without undue experimentation.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle.

In certain other embodiments, the compositions of the invention are formulated using one or more pharmaceutically acceptable excipients or carriers. In other embodiments, the pharmaceutical compositions of the invention comprise a therapeutically effective amount of a compound of the invention and a pharmaceutically acceptable carrier. In yet other embodiments, the compound of the invention is the only biologically active agent (i.e., capable of treating or preventing diseases and disorders related to aging) in the composition. In yet other embodiments, the compound of the invention is the only biologically active agent (i.e., capable of treating or preventing diseases and disorders related to aging) in therapeutically effective amounts in the composition. In yet other embodiments, the compound of the invention is co-administered with one or more addition biologically active agents (i.e., capable of treating or preventing diseases and disorders related to aging).

In certain other embodiments, the compositions of the invention are administered to [symbol] in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain other embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated in the invention.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as [symbol] binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

### Controlled Release Formulations and Drug Delivery Systems

In certain other embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds useful within the methods of the invention may be administered in the form of microparticles, for example by injection, or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, about 10 minutes, or about 1 minute and any and all whole or partial increments thereof after drug administration.

### Dosing

The therapeutically effective amount or dose of a compound of the present invention depends on the age, sex and weight of the patient, the current medical condition of the patient and the progression of a disease or disorder contemplated in the invention. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 5 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the disease or disorder, to a level at which the improved disease is retained. In certain other embodiments, patients require intermittent treatment on a long-term basis upon any recurrence of symptoms and/or infection. In other embodiments, compounds are administered continuously throughout the lifespan of the subject, regardless of health or disease state.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 5 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Methods of Screening

The invention further provides methods of rapidly and efficiently determining whether a compound extends the lifespan of a subject. In certain embodiments, the method [symbol] the use of modified “mother enriched” yeast cells wherein the yeast is modified such that the replicative capacity of the modified cells is not hampered while the replicative capacity of their progeny (second generation) cells is restricted.

In a non-limiting example, “mother enriched” yeast cells are cultured in a growth medium containing sufficient nutrients for cell growth and replication. The “mother enriched” yeast cells are labeled with an N-Hydroxysuccinimide (NHS) functionalized fluorophore, and then separated into equivalent aliquot samples. The samples are placed into sample wells in an array, and then treated with β-estradiol. The contents of each well is contacted with a test compound or control compound, and the resulting system is incubated for a period of time. Each sample is treated with a solution comprising a wheat germ agglutinin (WGA) functionalized fluorophore, such as CF405M-WGA, and a cell viability dye, such as propidium iodide, and then analyzed by flow cytometry to detect fluorescence from at least one fluorophore selected from the group consisting of the NHS functionalized fluorophore, WGA functionalized fluorophore and the cell viability dye. According to this non-limiting example, NHS functionalized fluorophore labeled cells are progenitor “mother enriched” yeast cells, while unlabeled cells are second generation cells. Further, cell viability dye labeled cells are determined to be dead cells or living cells, depending on the cell viability dye used. In certain embodiments, both a live cell staining dye and a dead cell staining dye are used simultaneously. In other embodiments, only one of a live cell staining dye and a dead cell staining dye are used. Further, WGA functionalized fluorophore labeled cells are cells that have replicated, while unlabeled cells are cells that have not replicated. In other embodiments, the WGA functionalized fluorophore selectively binds to “bud scars” on the “mother enriched” yeast cells and the intensity of the WGA functionalized fluorophore labeling corresponds to the number of replicative cycles a given cell has completed. In yet other embodiments, the number of bud scars can be observed, indicating the number of replications a “mother enriched” yeast cell has undergone. The WGA functionalized fluorophore, NHS functionalized fluorophore and cell viability dye can be detected using a variety of techniques, including but not limited to microscopy or fluorescence spectrometry.

In certain embodiments, the NHS functionalized fluorophore is at least one selected from, but not necessarily limited to, the group consisting of NHS-Fluorescein, NHS-Rhodamine, NHS-boron-dipyrromethene, sulfo-NHS-LC-Biotin, NHS-cyanine, NHS-benzopyrillium, and any of the NHS functionalized DYLIGHT™, ALEXA FLUOR™, EZ-LINK™, and PHRODO™ dyes available from ThermoFisher Scientific (Waltham, Mass.).

In certain embodiments, the cell viability dye is at least one selected from, but not [symbol] limited to, the group consisting of propidium iodide, phloxine B, methylene blue, rhodamine B, rhodamine 123, fluorescein diacetate, trypan blue, 7-aminoactinomycin D, SYTO 9, CFDA, Thiazole Orange, concanavalin A functionalized fluorophores, FUN-1® ((E)-2-((2-chloro-1-phenylquinolin-4(1H)-ylidene)methyl)-3-methyl-3l4-benzo[d]thiazole iodide), any of the MITOVIEW™ viability dyes (BIOTIUM), any of the LIVE-OR-DYE™ viability dyes (BIOTIUM), any of the LYSOVIEW™ viability dyes (BIOTIUM), and any of VIAFLUOR® viability dyes (BIOTIUM). In other embodiments, cell viability is determined using any commercially available dye, stain or cell viability assay known in the art, such as, but not limited to Cell Counting Kit-8 (Sigma-Aldrich) and BACTTITER-GLO™ Microbial Cell Viability Assay (Promega).

In certain embodiments, the WGA functionalized fluorophore is at least one selected from, but not necessarily limited to, the group consisting of Horseradish Peroxidase-WGA (HRP-WGA), CF®405M-WGA (BIOTIUM), CF®350-WGA (BIOTIUM), CF®405S-WGA (BIOTIUM), CF®488A-WGA (BIOTIUM), CF®532-WGA (BIOTIUM), CF®555-WGA (BIOTIUM), CF®568-WGA (BIOTIUM), CF®594-WGA (BIOTIUM), CF®633-WGA (BIOTIUM), CF®640R-WGA (BIOTIUM), CF®680-WGA (BIOTIUM), CF®680R-WGA (BIOTIUM), and CF®770-WGA (BIOTIUM). The CF® family of fluorophores are described in U.S. Pat. Nos. 8,436,170 B2, 8,658,434 B2, 9,097,667 B2, and 9,579,402 B2 which are incorporated herein by reference in their entirety. In other embodiments, the WGA functionalized fluorophore is any WGA functionalized fluorophore known in the art. In yet other embodiments, other fluorophores that selectively or preferentially bind to bud scars are used in place of the WGA functionalized fluorophore, such as Calcofluor White.

The present methods allow for the determination of the mean lifespan of the yeast cells in a sample. In certain embodiments, the samples are analyzed by flow cytometry at multiple time points in order to monitor mean lifespan over a period of time. In other embodiments, the yeast cells are monitored by flow cytometry over a period of time with samples taken at time points between 0 hours and about 48 hours.

In certain embodiments, the yeast cells are cultured in a growth medium comprising complete supplement mixture (CSM) and glucose. In other embodiments, the growth medium comprises at least one nutrient selected from the group consisting of Adenine, L-Arginine, Glucose, L-Aspartic acid, L-Histidine HCl, L-Isoleucine, L-Leucine, L-Lysine HCl, L-Methionine, L-Phenylalanine, L-Threonine, L-Tryptophan, L-Tyrosine, Uracil and Valine.

In certain embodiments, the yeast cells are cultured in air. In other embodiments, the [symbol] re cultured at a temperature of about 30° C. In yet other embodiments, the yeast cells are incubated in the array for a period of time selected from the group of about 0, 8, 24, 32, about 48 hours and any time there between.

In certain embodiments, the flow cytometry is automated flow cytometry. In other embodiments, the array is a multi-well plate comprising a plurality of sample wells. In yet other embodiments, the array is a 384-well plate.

### Kits

The invention further provides kits comprising materials necessary to carry out the screening methods of the invention.

The kit can comprise at least one vessel adapted and configured for culturing yeast. The kit can comprise a growth medium for culturing yeast. The kit can comprise genetically modified “mother enriched” yeast. The kit can comprise at least one selected from the group consisting of NHS-Fluorescein, β-estradiol, CF405M-WGA and propidium iodide.

In certain embodiments, the kit comprises instructional materials comprising instructions for carrying out the screening methods of the invention.

In certain embodiments, the kit further comprises at least one multi-well plate. In other embodiments, the multi-well plates are adapted and configured for use with an automated flow cytometer.

In certain embodiments, the growth medium comprises complete supplement mixture (CSM) and glucose. In other embodiments, the growth medium comprises at least one nutrient selected from the group consisting of Adenine, L-Arginine, Glucose, L-Aspartic acid, L-Histidine HCl, L-Isoleucine, L-Leucine, L-Lysine HCl, L-Methionine, L-Phenylalanine, L-Threonine, L-Tryptophan, L-Tyrosine, Uracil and Valine.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the [symbol] in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed, proguanil all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### Yeast Strains, Media, and Culture Conditions

All experiments were conducted in a BY4741 strain background (TransOMIC TKY0002). Strains containing the genetic modifications of the Mother Enrichment Program (MEP) (Lindstrom, et al., Genetics. 2009 October; 183(2):413-22) were constructed by lithium acetate transformation (Gietz, et al., Nat. Protoc. 2, 31-34 (2007)) with PCR products derived from MEP strain UCC8773. Deletion strains were prepared similarly, with transformation DNA from PCR on the genomic DNA of corresponding strains from the yeast deletion library (Giaever et al., Nature 418, 387-391 (2002)) (GE Dharmacon).

Synthetic media (CSM 2% glucose) was used for all experiments. Cells were maintained in aerobic conditions at 30° C., in either 50 mL conical tubes (Becton Dickinson F2070) or 384-well plates (Greiner Bio-One 781201). Cultures in tubes were performed in [symbol] 2 shaker (New Brunswick Scientific) at 225 rpm. Plate-based cultures were performed in a humidified incubator kept at 95% relative humidity, and the plates were covered with a breathable membrane (Thermo Scientific 241205) to prevent evaporation. Agitation was provided by a microplate shaker (Union Scientific 9779-TC) at an amplitude of 0.04 inches.

### Compounds for High-Life Screening

As a positive control for lifespan extension, ibuprofen (Sigma 1-1892) was used. The following compound libraries, obtained from the Yale Center for Molecular Discovery, were screened: (1) 320/355 compounds in the Selleckchem Kinase Inhibitor Library, (2) the Enzo-640 FDA-approved drugs catalog, (3) the Enzo Kinase Inhibitor Library, and (4) the Microsource Pharmakon 1600 library.

### Determining Maximum Cell Density to Avoid Nutrient Depletion

10 mL of cells were grown overnight for 16 hours to mid-log phase, then diluted to the indicated densities in ice-cold media. 80 μL of cell suspension was aliquoted to 12 wells of four 384-well plates for each cell density. The plates were covered with a breathable membrane and placed on a shaking platform in a humidified incubator for 3 hours. Each well was then treated with 20 μL pre-warmed, 30° C. 5 μM β-estradiol (Sigma E8875) in media, and the plates were returned to the incubator. For the 0-hour timepoint, this addition was instead performed immediately after initially aliquoting the plate. After 0, 8, 24, and 48 hours from the time cells were aliquoted to the plate, the total cell count was measured using a flow cytometer.

### NHS-Fluorescein Labeling

10 mL of cell culture was grown overnight for 18 hours to mid-log phase. The cells were then spun down at 1000×g for 3 minutes at room temperature. The supernatant was poured off, and the cells were re-suspended in 1 mL 3.5 mg/mL NHS-Fluorescein (Life Technologies 46410) in 10×PBS (Life Technologies 14200075). The cells were then placed on a rocking platform in the dark for 15 minutes at room temperature. The cells were then diluted to 50 mL in ice-cold 1×PBS (Life Technologies 141901144), mixed, and spun down at 1000×g for 2 minutes at 4° C. The supernatant was discarded, and this wash step was repeated. Afterward, the supernatant was discarded, and the cells were re-suspended in 1 mL ice-cold media.

### Measuring the Effect of Labeling on Cell Health

10 mL of cells were grown for 16 hours overnight to mid-log phase. 1 mL of cells were aliquoted to a fresh tube and placed on ice as the unlabeled control. The remaining 9 mL were labeled with NHS-fluorescein as described above. Cells were diluted in media containing 1 μM β-estradiol to 5 cells/μL, and 500 μL of cell suspension was aliquoted to flow-cytometry tubes and placed in a 30° C. shaking incubator. After 0, 8, 24, 32, and 48 hours, a set volume was acquired for three tubes per timepoint for each condition using a BD FACSVerse flow cytometer. Cell count was then normalized to the 0-hour timepoint.

### High-Life Experiments

Cells were labeled with NHS-fluorescein as described above, then diluted to 20 cells/μL in media. 80 μL/well was aliquoted to 384-well plates, which were covered with a breathable membrane and placed on a shaker platform in a humidified incubator. After 3 hours, the plates were removed from the incubator and each well was treated with 20 μL pre-warmed, 30° C. 5 μM β-estradiol in media. In the case of ibuprofen or compound-treated wells, the compound was diluted in this volume at a 5× concentration to achieve a final concentration of 10 μM, or 100 μM for ibuprofen. The plates were then returned to the incubator. At indicated times, one plate was removed from the incubator and placed on an autosampler cooled to 8° C. attached to a Stratedigm flow cytometer. The cytometer was set to automatically add and mix 20 μL of aqueous solution containing 60 μg/mL CF405M-WGA (Biotium 29028) and propidium iodide (Sigma P4864) prior to acquiring 80 μL of sample for each well.

### Replicator Experiments

To obtain the single-cell level data for the age and generation-durations of replicatively aging mother cells, data reported in Liu, P., et al., Cell Rep. 634-644 (2015) was re-analyzed. The data was collected in the same media condition (CSM 2% glucose) as reported elsewhere herein. Cells were grown for ˜24 hours in CSM 2% glucose prior to loading to the microfluidic device. Once cells were loaded, media was swapped to provide CSM 2% glucose control media (untreated), media containing DMSO as a vehicle control (American Bio AB00435), media containing 10 μM terreic acid (Sigma SML0480), media containing 10 μM mycophenolic acid (Sigma M5255), or media containing another compound at the indicated concentration. An automated microscope was used to track [symbol] another cells, and replicative lifespan was later determined by counting the number of daughters produced before death. In the case of compound validation, only newborn cells were included in the lifespan experiment. To compare the replicative lifespan of labeled and unlabeled cells, only cells that were present within the traps at the start of the experiment were included. For these experiments, cells were loaded to the microfluidic device at an increased rate of 100 μL/min to increase the number of trapped mother cells. Green fluorescent images were also taken at the start of the experiment to confirm that the cells were visibly labeled.

### Bud Scar Staining

Cells were prepared as described in the “High-Life Experiments” section above, except diluted to 100 cells/μL prior to loading on a plate. After 0, 8, and 24 hours, all cells from a single plate were transferred to a 50 mL conical tube, and pelleted at 1000×g for 3 minutes. The supernatant was aspirated, and the cells were resuspended in 900 μL of sterile water and transferred to a 1.5 mL tube. 100 μL of 1 mg/mL Fluorescent Brightener 28 (Sigma F3543) in water was added, and the solution was incubated at room temperature in the dark for 5 minutes. Next, the solution was pelleted at 13000×g for 30 seconds, the supernatant was aspirated, and the cells were resuspended in 1 mL sterile water. This wash step was then repeated once. The cells were resuspended in 10 μL sterile water, and stored on ice in the dark until imaged. Z-stack brightfield and fluorescent images with 0.2 μm spacing were acquired for each sample on a confocal microscope. For green-fluorescent mother cells, the number of bud scars in the blue fluorescent channel were counted manually.

### Confidence Interval Determination and Computing Areas Between Curves

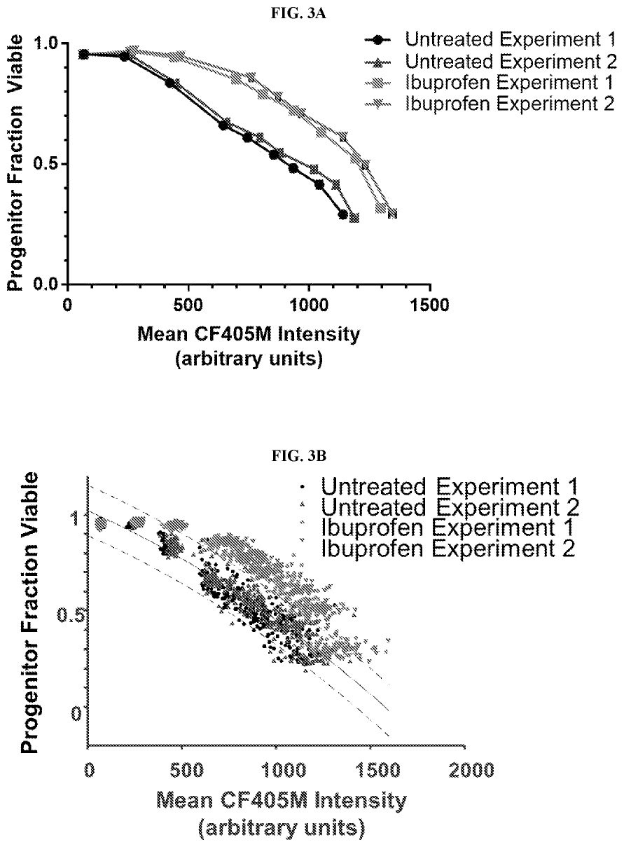

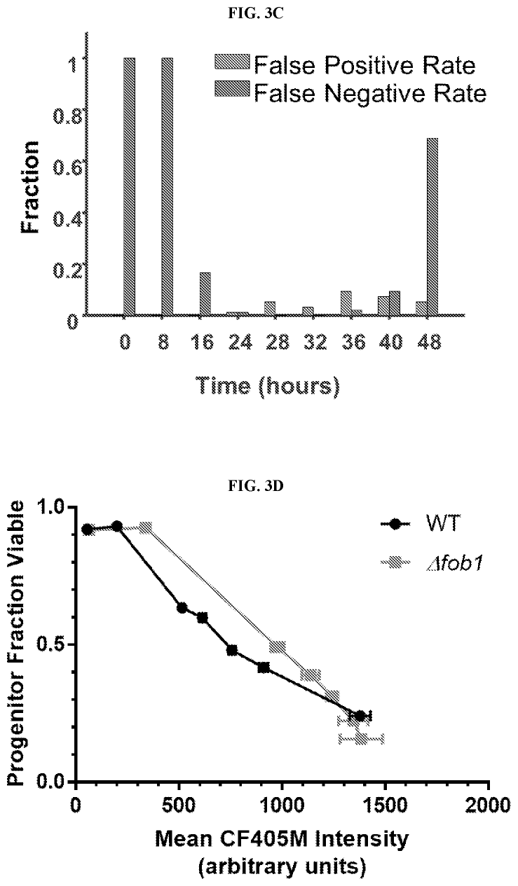

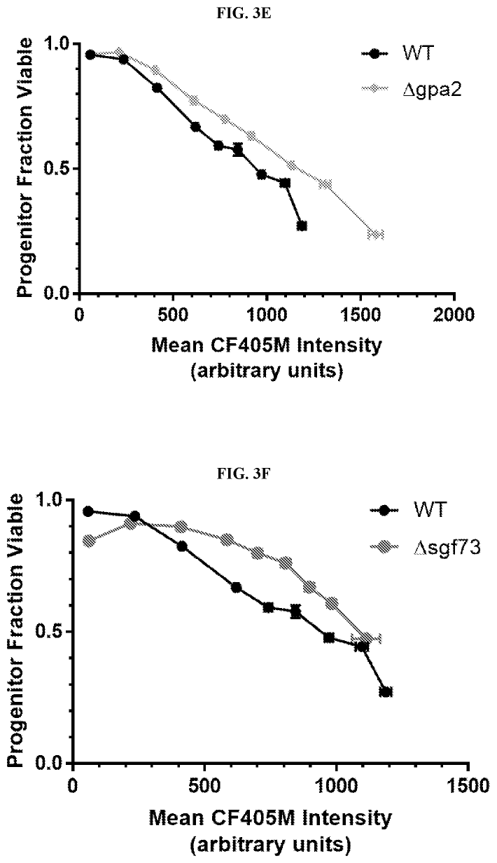

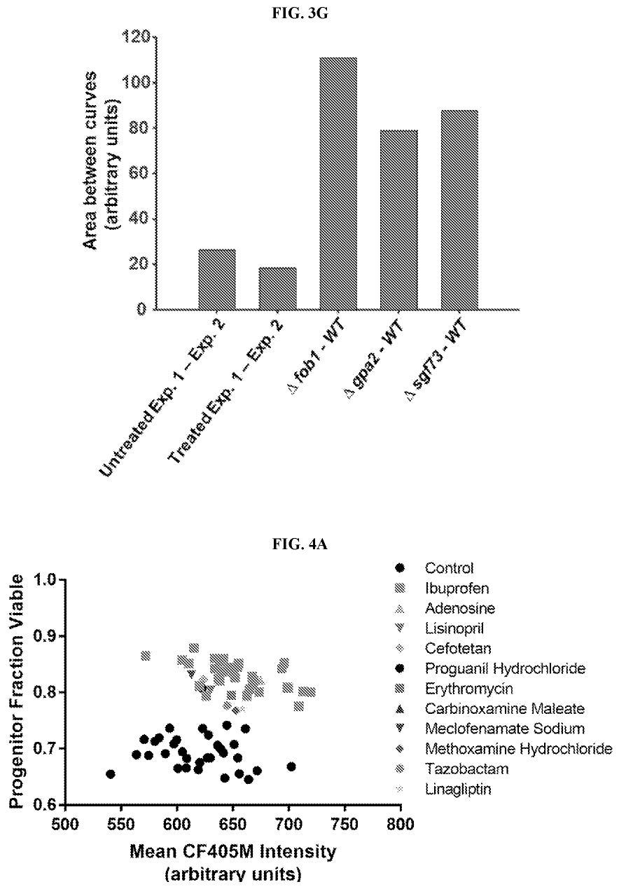

In FIG. 3B, second-order polynomial fitting was performed on the pooled data set obtained from the two untreated experiments. Both the fitting and the 95% confidence interval computation were performed using MATLAB's curve fitting toolbox.

Areas under respective curves were computed using trapezoidal numerical integration by calling MATLAB's trapz function. The numerical integrations were performed over the same Mean CF405M Intensity range from 70 to 1100. Corresponding fractions of progenitor cells viable at the starting (70) and the ending point (1100) were computed through linear interpolation for each curve. Area differences between respective pairs of curves were then computed to generate FIG. 3G.

[symbol]

action and Proteasome Assay

For protein extraction, 50 mL of cells were grown for 18 hours to an OD600 of approximately 0.8, then transferred to a 50 mL conical tube and centrifuged at 4255×g for 5 minutes at room temperature. The supernatant was discarded, and the cells were re-suspended in 150 μL of cold lysis buffer (50 mM Tris-HCl, pH 7.5, 0.5 mM EDTA, 5 mM MgCl₂, with complete ULTRA mini protease inhibitor tablets, EDTA free) and transferred to a 1.5 mL tube. A ¼ volume of 500-750 μm glass beads (Acros Organics 397641000) was added to each tube. For 10 rounds, the tubes were chilled in ice water for 1 minute, then vortexed at maximum speed for 30 seconds to physically rupture the cells, and returned to the ice water. Samples were then spun for 3 minutes at 2500×g and 4° C., and the supernatant was transferred to a fresh tube. The solution was further clarified by centrifugation at 8000×g for 10 minutes at 4° C., and the supernatant was transferred to a fresh tube. Protein concentration was measured using a Nanodrop measuring the absorbance of the sample at 280 nm.

The proteasome assay used was described in Kruegel, et al. PLoS Genet. 7, (2011). The assay was performed in a 96-well clear-bottom plate (Costar 3603) with 50 μg of total protein in 200 μL of lysis buffer. The fluorogenic proteasome substrate Suc-LLVY-AMC (Bachem 1-1395) was added to a final concentration of 100 μM. Fluorescence intensity with an excitation wavelength of 380 nm and an emission wavelength of 460 nm was recorded at 5-minute intervals using a Neo2 plate reader (BioTek) set to mix constantly and maintain 30° C. Negative control reactions were performed in the presence of 50 μM MG132 (Sigma 474787), a proteasome inhibitor.

### Statistical Methods

Differences in lifespan characteristics were assessed through Log-Rank test using MATLAB with a cut-off value of P=0.05. The script for Log-Rank test was downloaded from MATLAB. Differences in proteasome activity were assessed using the unpaired, two-tailed, parametric t-test function of the GraphPad Prism software.

### Logic and Limitations of the Longevity Placement Test

The Longevity Placement Test (LPT) was designed as a mutually exclusive, collectively exhaustive test to determine the relationship of a longevity intervention to a known genetic regulator of lifespan. For a given intervention that extends lifespan, any of three possible relationships may exist relative to a known genetic regulator of lifespan. (1) The intervention may act to extend lifespan independently from the known regulator. (2) The [symbol] may act downstream from the known regulator, converging on a single component of the known regulator's lifespan pathway. (3) The intervention may act upstream from or upon the known regulator, ultimately modulating lifespan through the genetic regulator.

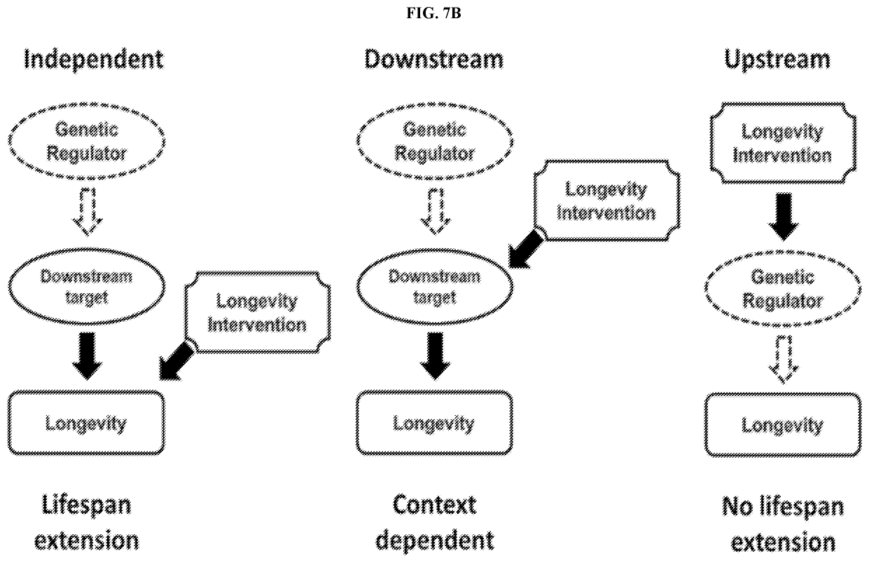

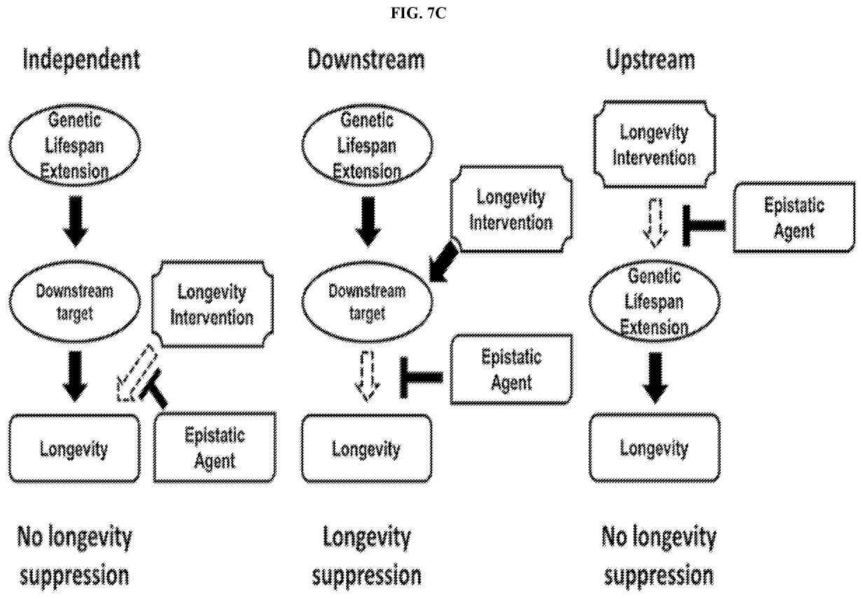

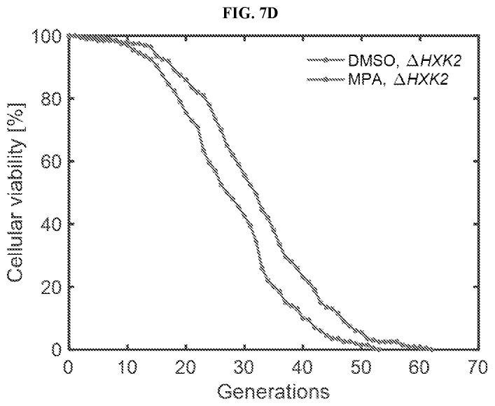

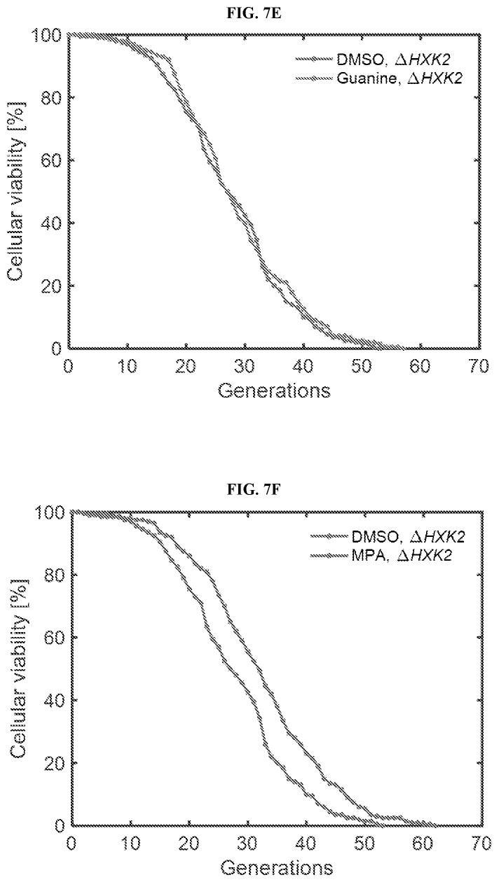

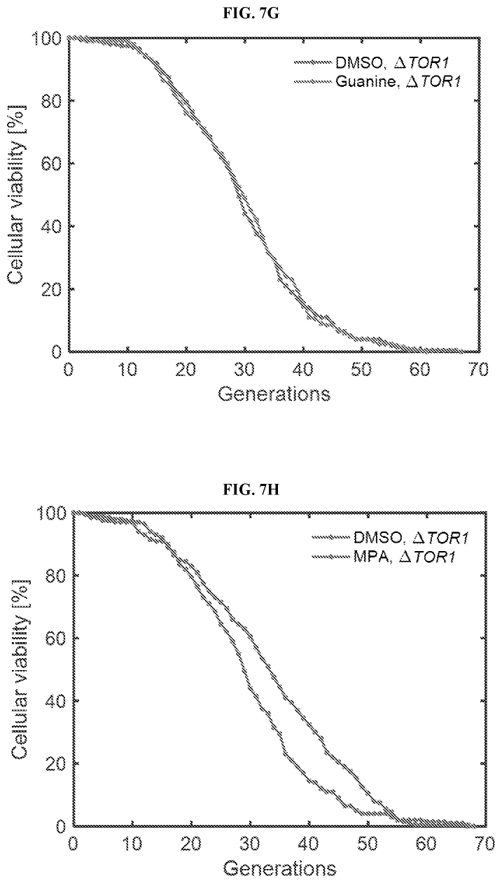

In Step 1 of the LPT, the longevity intervention is applied to a strain in which a genetic regulator of lifespan is deleted. In the event that lifespan extension from the longevity intervention is observed in this background, only possibilities (1) and (2) above remain valid (FIG. 7B). If no lifespan extension is observed, then possibilities (2) and (3) remain valid. Possibility (2) cannot be ruled out in this step, since non-saturating action by the genetic regulator could leave room for lifespan extension by the longevity intervention, while saturating action would preclude it.

In Step 2 of the LPT, an epistatic agent, which prevents lifespan extension from the longevity agent, is applied to a strain in which some upstream member of the genetic regulator's lifespan pathway has been modified to extend lifespan. This step differentiates possibility (2) from the remaining possibility after Step 1. In the event that lifespan extension from the genetic regulator's pathway is suppressed by the epistatic agent, this determines that possibility (2) is correct. In the event that no epistasis is observed, the remaining possibility, (1) or (3), is correct (FIG. 7C).

Preconditions, both physical and experimental, exist for an LPT experiment to conclusively relate a longevity intervention to a given pathway. Two physical factors must exist for an exhaustive LPT test: a longevity agent to test, and an epistatic agent that prevents lifespan extension from the longevity agent. Importantly, the epistatic agent must not directly affect the longevity agent, such as through inactivating it. Ideally, it should exert an opposing effect on some downstream target, ensuring that its suppression will be generalizable to any actor upstream of the longevity agent's target. There also exist constraints on the choice of genetic manipulations for investigation. For Step 1, probe gene deletion should not shorten RLS, as this may mask longevity effect. For Step 2, the intervention which extends RLS must act upon or upstream from the Step 1 probe gene in order to create complete coverage of the pathway. However, the intervention in Step 2 need not always be a gene deletion; for example, in the case of SIR2, deletion of SIR2 in Step 1 could be complemented by overexpression of SIR2, an intervention which extends lifespan, in Step 2.

Roundworm (C. elegans) Lifespan Extension Procedures

Synchronized animals were obtained using the egg-laying method, allowing young [symbol] eggs for 4 hours on bacteria-seeded plates. For each treatment group, 120 synchronized day-1 adults were used. All experiments were carried out at 20° C. Treatment groups were blinded. Nematode growth media (NGM) plates were made for each treatment group: Negative control (DMSO), Mycophenolic Acid (10 μM in DMSO), and Terreic Acid (10 μM in DMSO). The compounds were added to NGM media before pouring the plates. The plates were then dried overnight before moving them to 4° C. to prevent degradation of the compounds. Plates were seeded with 100 μL of 10× concentrated OP50 E. coli, and dried overnight at room temperature. Plates were UV treated with a UVP CL-1000 Ultraviolet Crosslinker, run twice on the energy setting ‘9999’ for about 5 minutes each run. Worms were transferred to new plates every 2-4 days. Viability was scored every day, with death determined by lack of response to a platinum wire. Missing worms, or those that died due to internal hatching were censored.

### Example 1: High Throughput Replicative Lifespan Measurement (High-Life)

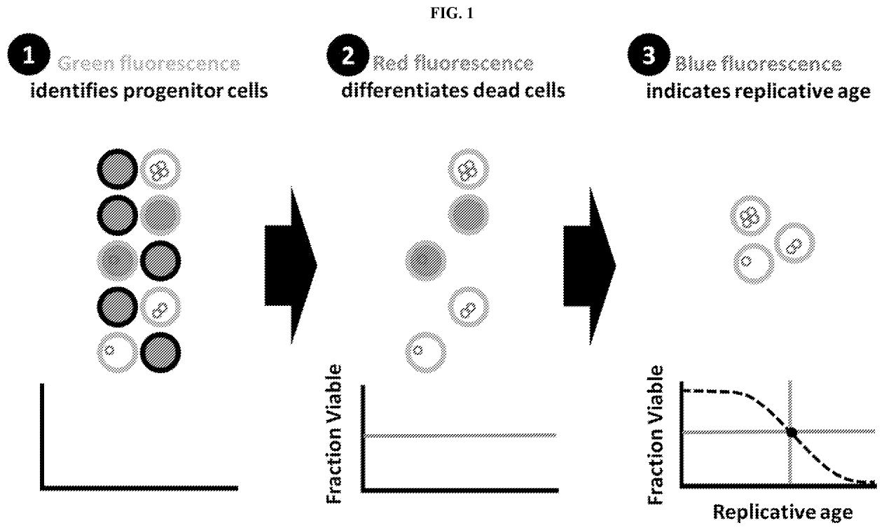

In order to test the lifespan of model organisms on both a large-scale and with quick turn-around, a massively multiplexed method was developed to measure replicative lifespan in the short-lived model organisms Saccharomyces cerevisiae. The protocol uses green-fluorescent labeling to identify progenitor cells, red-fluorescent labeling to differentiate non-viable cells, and blue-fluorescent labeling of bud scars to determine replicative age (FIG. 1). Each parameter is measured using a flow cytometer. Using a plate-based autosampler, throughput is >1000 wells per day, each containing a different strain or media condition.

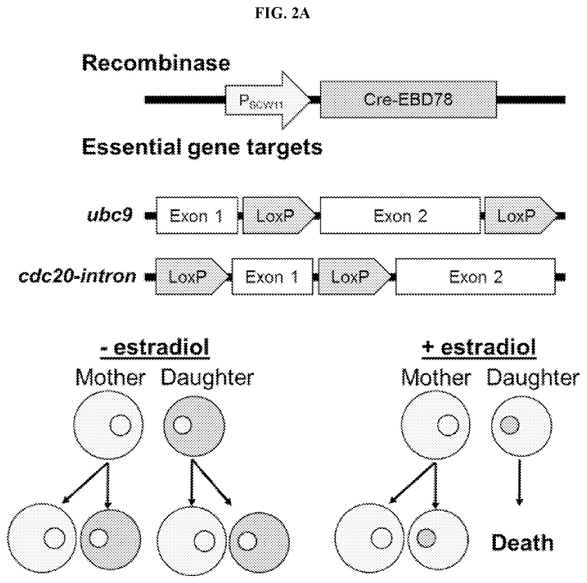

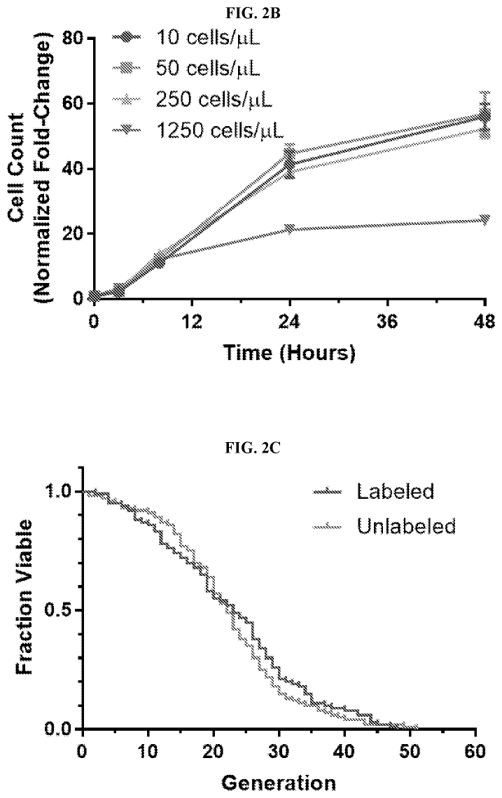

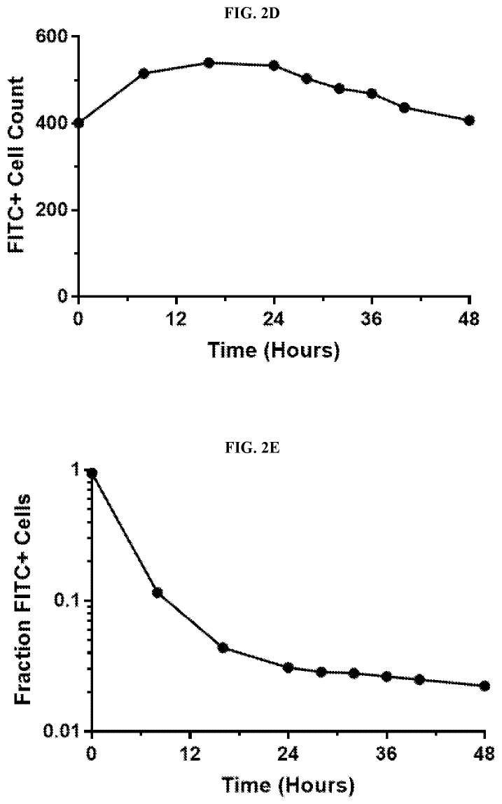

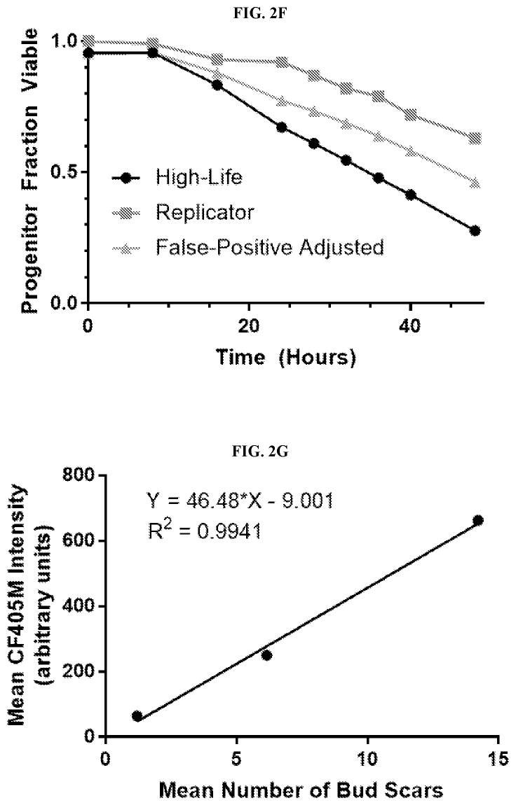

To achieve high throughput, the measurement system was automated, and the assay was performed in 384-well plates. The assay was performed using an autosampler-equipped flow cytometer, in a volume of 100 μL. Growth of even a single cell and its progeny in such a small volume will result in nutrient starvation before the natural replicative lifespan is exhausted; therefore to circumvent this issue, High-Life experiments were performed in the background of the Mother Enrichment Program (MEP) (Lindstrom and Gottschling, Genetics. 2009 October; 183(2):413-22). MEP strains express a CRE recombinase fused to an estrogen-binding domain for only a short time after birth. In the presence of β-estradiol, the recombinase translocates to the nucleus where it can excise two essential genes that have been modified to contain the exogenous LoxP sequence. Addition of β-estradiol to the media thus renders newborn daughters inviable without affecting existing mother cells, preventing exponential growth of the cell population and nutrient depletion (FIG. 2A).

Throughput of a flow-cytometry based assay is dependent on the cell density, as cell [symbol] negatively correlated with the sample processing speed in each well. In order to process the entire 384-well plate as fast as possible, the maximum cell density which could be used without causing nutrient depletion was determine. The MEP was induced with β-estradiol and the cells were cultured at different densities, then the total cell number was measured at various times up to 48 hours later. No growth rate defect was observed for inoculation densities of up to 250 cells/μL (FIG. 2B). To reduce the risk of partial nutrient depletion, subsequent experiments were performed with <20 cells/μL.

Replicative lifespan has two fundamental parameters: replicative age in the population of interest and the fraction of cells viable at that age. These lifespan parameters were measured in an unmonitored liquid culture in three steps: (1) differentiation of the progenitor cells of interest from their progeny, (2) identification of the viable fraction of progenitor cells, and (3) determination of the replicative age of the viable progenitor cells.

Asymmetric segregation of the cell wall between mother and daughters enables magnetic sorting of a progenitor cell population (Smeal, et al. Cell 84, 633-642 (1996)). This technique was used to label the progenitor population cell wall with a fluorescein conjugated N-HydroxySuccinimide-ester (NETS-ester) (FIG. 1, step 1). The label itself did not alter the cells' natural lifespan. This was tested by using total cell count as a measure of replicative capacity. No growth rate change was observed in labeled cells compared to unlabeled controls, indicating the procedure did not affect replicative capacity or cell health (FIG. 2C). It was also confirmed that the fluorescent label was retained by mother cells, and not passed to their daughters. When cultured, the total number of labeled cells (mothers) increased after initiation, and subsequently declined gradually (FIG. 2D). The initial increase in labeled cells is consistent with separation of cells that were partially budded during labeling; the decline can be explained by fragmentation of dead cells such that they no longer triggered the flow cytometer. A decrease in the fraction of labeled cells over time (FIG. 2E) was also observed, representing the generation of unlabeled daughters. Overall, these results indicate that the population of progenitor cells in an unmonitored liquid culture was able to be tracked without impacting cell health.

Once the progenitor cell fraction was identified, the viable fraction of the cells was determined (FIG. 1, step 2). The viability dye propidium iodide was used in order to label, culture, and stain the cells. Flow cytometry revealed a time-dependent decline in progenitor cell viability, consistent with expectations for an aging population (FIG. 2F). To assess if the rate of decline was the same as observed using other lifespan measurement methods, a medium-throughput, single-cell Replicator device (Liu, P., et al. Cell Rep. 634-644 (2015)) [symbol] This technology allowed for the collection of images of trapped mother cells throughout their entire lifespan. Analyzing the image series, replicative lifespan and the length of each budding interval was measured. The rate of viability decline was measured with propidium iodide, and it was observed that the rate of decline exceeded that seen in the Replicator device experiments (FIG. 2F). The Replicator device was then used to measure fluorescence intensity of cells introduced to propidium iodide after 16 or 40 hours in culture. After 16 hours, 2% of live cells fell above an intensity threshold constructed to approximate the flow cytometry experiment's gate. By 40-hours, 20% of live cells fell into the dead region, largely due to dead daughter cells that failed to separate, indicating that a small fraction of aged but live cells stain as non-viable using propidium iodide. Based on an assumption of linearity between these points, a false-positive rate over the entire time-course of a High-Life experiment was projected, and a corrected viability curve was plotted (FIG. 2F). The curve did not precisely match that observed using the Replicator device, suggesting that limitations in the intrinsic ability to relate microscopic fluorescence intensity data to flow cytometric data may underlie the remaining difference.

Next, the replicative age of the viable progenitor cells was measured (FIG. 1, step 3). A bud scar is left on the mother cell wall with each division, and therefore the number of bud scars is directly proportional to replicative age. The protein lectin wheat germ agglutinin (WGA) has been demonstrated to bind specifically to bud scars. WGA conjugated to the blue fluorophore CF405M was used to measure replicative age throughout the lifespan by labeling cells, culturing, and staining simultaneously with CF405M-WGA and propidium iodide. The CF405M intensity measured with a flow cytometer was compared to the number of bud scars observed microscopically on cells cultured for the same period of time, a proportional relationship was observed (R²=0.9941) (FIG. 2G), confirming the ability to measure replicative age with this method.

### Example 2: Testing of Life Extending Compounds

High-Life experiments were conducted in the presence and absence of ibuprofen. Cells were labeled, cultured in the presence of β-estradiol and +/− ibuprofen and stained with propidium iodide and CF405M-WGA at multiple later time points. Readings were then acquired with a flow cytometer. An increase in replicative lifespan was observed in the presence of ibuprofen, confirming the ability of the High-Life method to detect lifespan extension (FIGS. 3A-3B). To assess the sensitivity and specificity of High-Life, a trend-line and 95% confidence interval were fit to the untreated condition (FIG. 3B). Using this [symbol] interval as a cut-off, the fraction of ibuprofen-treated samples that fell within the confidence interval (false-negatives) and fraction of untreated samples that fell outside the confidence interval (false-positives) were measured for each timepoint (FIG. 3C). The rate of false-negatives and false-positives was found to be the lowest for measurements taken after 24-hours of culture, and this length of culture was used for comparative measurements of replicative lifespan.

After testing this technique in the ibuprofen environment, the High-Life method was used to identify increases in lifespan from genetic interventions. The technique was used to create replicative lifespan curves for three strains harboring gene deletions previously demonstrated to extend replicative lifespan: fob1, gpa2, and sgf73. All three strains showed an increase in replicative lifespan compared to the wild-type control (FIGS. 3C-3E). The difference between wild-type and long-lived strain measurements was then quantitively assessed. For this purpose, the area between curves in the wild-type, ibuprofen-treated, and long-lived strain conditions was measured (FIG. 3G). The area was 3-5× greater when comparing conditions with an expectation for a lifespan difference, versus for two experiments performed for the same condition. The data indicated that extension of replicative lifespan could reliably and reproducibly be detected using the High Life techniques.

### Example 3: Identification of Lifespan Extending Compounds

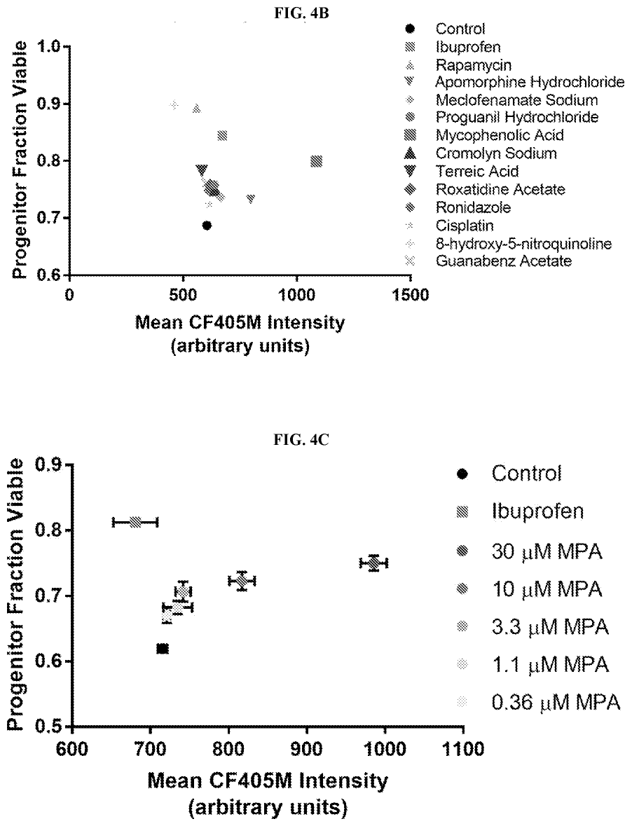

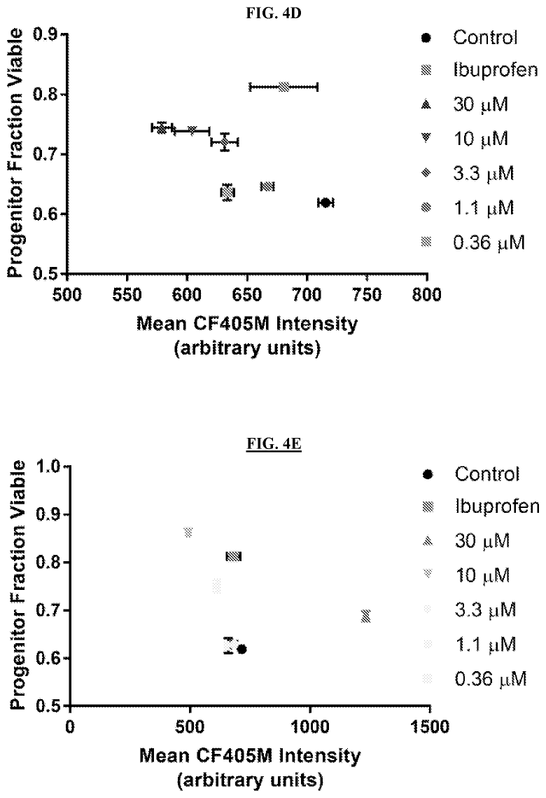

The High-Life method was then tested to determine if it was suitable to screening for compounds that extend replicative lifespan. A diverse library of 2640 compounds was selected, including kinase inhibitors, FDA-approved compounds, and compounds which had failed clinical development. The effect of these compounds on High-Life readings was assayed after 24 hours in culture at 10 μM concentration. As a positive control, ibuprofen was used. Replicates of ibuprofen treatment were reproducibly distinguishable from negative control points (FIG. 4A). To test the analytical specificity of the screen, 99 follow-up compounds were selected which qualitatively deviated from the control (FIG. 4A). The experimenters remained blinded to the identity of these compounds until the original results were repeated: a second 24-hour High-Life measurement was conducted with 3-4 replicate wells for each compound to differentiate random variation from genuine lifespan extension. The average readings for 12 compounds fell at least slightly above controls (FIG. 4B), and their identities were unblinded. These compounds included mycophenolic acid, terreic acid, rapamycin, guanabenz acetate, proguanil hydrochloride (or chloroguanide hydrochloride), [symbol] e hydrochloride, cromolyn sodium, meclofenamate sodium, roxatidine acetate hydrochloride, ronidazole, cisplatin, and nitroxoline.

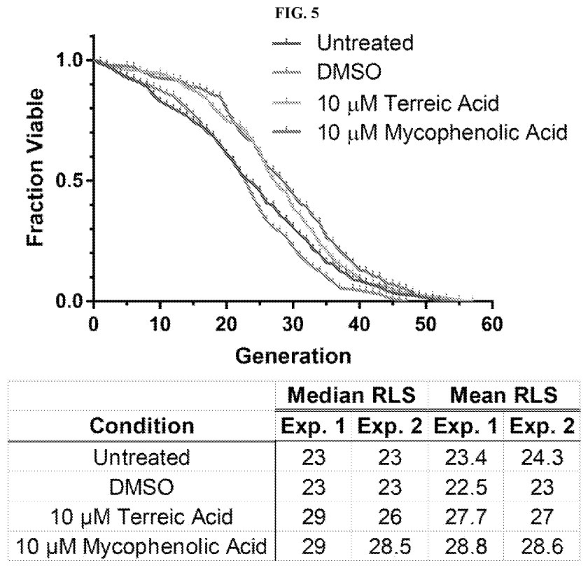

Fresh samples for 7 of these compounds were obtained from a secondary source, and subjected to a dose-response experiment. Three compounds exhibited concentration-dependent increase in cell survival (FIG. 4C), while the remainder continued to show only mild deviation from the control. To differentiate artifactitious High-Life readings from lifespan extension, secondary validation experiments were performed for the three compounds: RLS was measured on a single-cell level in the presence of 10 μM compound using the Replicator device (Liu, P., et al. Cell Rep. 634-644 (2015)). One compound, 8-hydroxy-5-nitroquinolone, was toxic and caused most cells to arrest immediately. However, terreic acid and mycophenolic acid exhibited 15% and 20% extension of mean RLS, respectively (FIG. 5).

### Example 4: Mechanistic Studies on Mycophenolic Acid Lifespan Extension

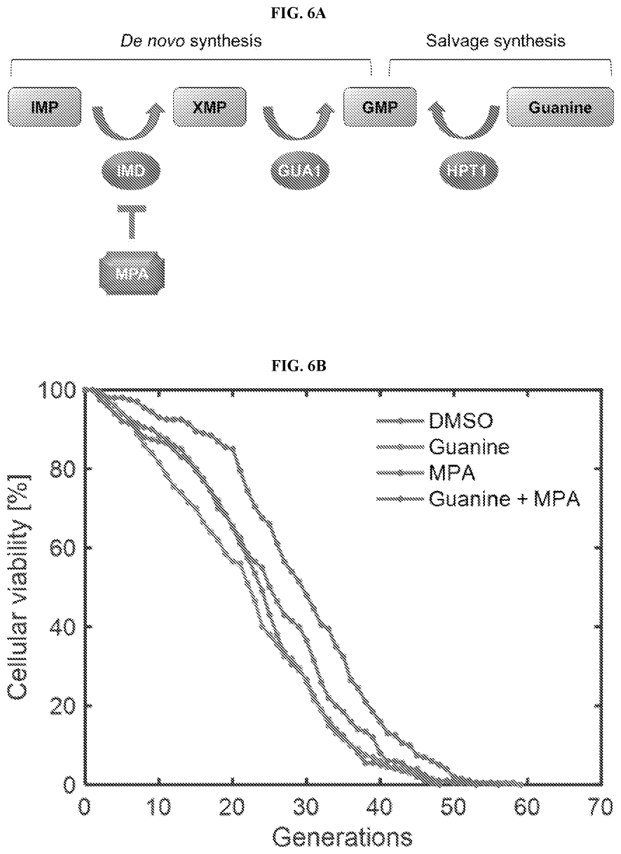

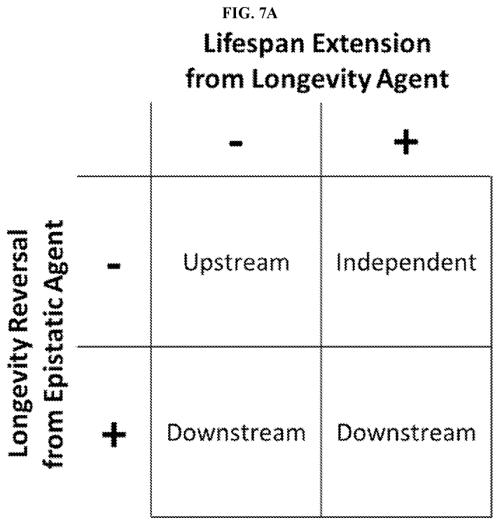

Mycophenolic acid (MPA) is known to reduce cellular guanosine monophosphate/guanosine triphosphate (GMP/GTP) pools through inhibition of inosine monophosphate dehydrogenase (IMD), the rate-limiting enzyme in de novo GMP synthesis (FIG. 6A). Without intending to be limited to any particular theory, it is possible that this mechanistic function is responsible for MPA's lifespan extending effect. GMP can also be synthesized via a salvage pathway in the presence of exogenous guanine. Therefore, replicative lifespan (RLS) was measured in the presence of MPA with and without supplemental guanine in order to determine if the longevity effect of MPA is prevented by exogenous guanine (FIG. 6B). It was found that MPA treated samples without supplemental guanine had extended longevity as compared to control and guanine supplemented samples. Without intending to be limited to any particular theory, these results suggest that MPA may extend RLS in S. cerevisiae through inhibition of GMP synthesis.

The role of GMP synthesis inhibition on lifespan extension was then investigated. A generalizable and systematic approach to categorize longevity interventions to genetic regulators of lifespan was developed. In theory, a longevity intervention can act either within or independent from a known longevity pathway. If within a longevity pathway, the intervention must act upon, upstream from, or downstream of a given pathway component. The placement of MPA relative to the known genetic lifespan pathways was identified using a two-step test, referred to herein as the “Longevity Placement Test” (LPT) (FIG. 7A-7C). In Step 1, it was determined if the longevity intervention extends lifespan in a strain lacking a [symbol] lifespan pathway component, the probe gene. In Step 2, it was determined whether an epistatic agent that prevents lifespan extension from the longevity intervention can also prevent lifespan extension conferred by modulation of the probe gene. By combining this information, the relationship of a longevity intervention to a known lifespan regulation pathway can be definitively classified.

The LPT system was used to determine the relationship of GMP depletion to the three major lifespan-extension pathways known for S. cerevisiae (Longo, et al., Cell Metabolism 16, 18-31 (2012).). MPA was used as the longevity intervention, and guanine was used as the epistatic agent. The first pathway tested was the nutrient sensing pathway, which encompasses dietary restriction and the target of rapamycin (TOR) inhibition. As the LPT probe genes, TOR1 and HXK2 were chosen, the individual deletion of which is known to extend yeast lifespan. TOR1 is a protein kinase subunit of the TORC1 complex that controls cell growth in response to nutrient availability; HXK2, on the other hand, is a hexokinase whose deletion provides a genetic mimicry of nutrient limitation because it phosphorylates intracellular glucose as part of glucose metabolism. MPA further extended lifespan in the long-lived ΔTOR1 and ΔHXK2 strains, while guanine did not suppress lifespan extension in these strains (FIG. 7D-7G and Table 1). Without intending to be limited to any particular theory, these results suggest that GMP depletion exerts its longevity effect independent of the nutrient sensing pathway.

The second lifespan-extension pathway tested to determine its relationship to GMP insufficiency was the sirtuin pathway. As the LPT probe gene, SIR2 was chosen, an evolutionarily conserved histone deacetylase, the deletion of which shortens yeast lifespan, while its overexpression extends lifespan. However, shortened lifespan in the ΔSIR2 background masks the effect of most longevity interventions because ΔSIR2 cells die from the rapid accumulation of rDNA circles before other aging factors accumulate (Delaney, et al., Aging Cell 10, 1089-1091 (2011).). This lifespan shortening and longevity masking can be rescued by concurrent deletion of FOB1, a nucleolar protein, the deletion of which reduces formation of rDNA circles (Defossez, et al. Mol. Cell 3, 447-455 (1999).). MPA was able to extend the lifespan in the absence of SIR2 in the ΔSIR2ΔFOB1 background (FIG. 7H) while guanine did not reverse the lifespan extension conferred by SIR2 overexpression (FIG. 7I). Without intending to be limited to any particular theory, these results suggest that GMP depletion extends lifespan independent of the sirtuin pathway.

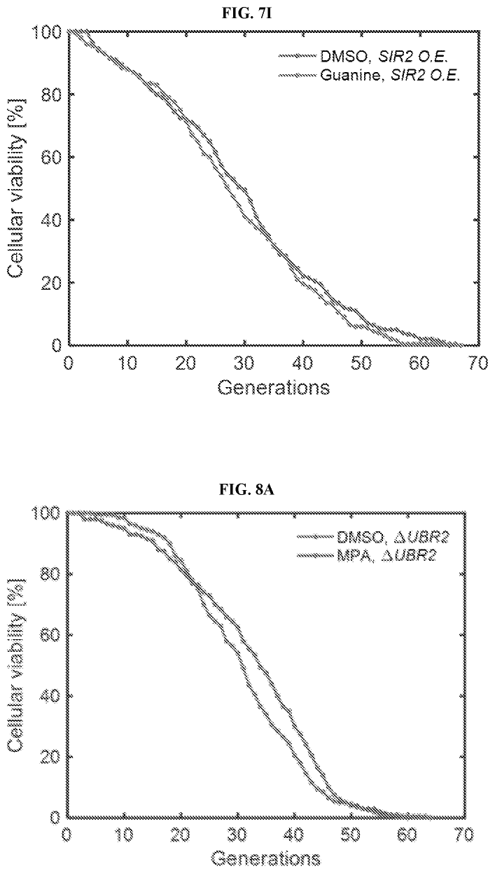

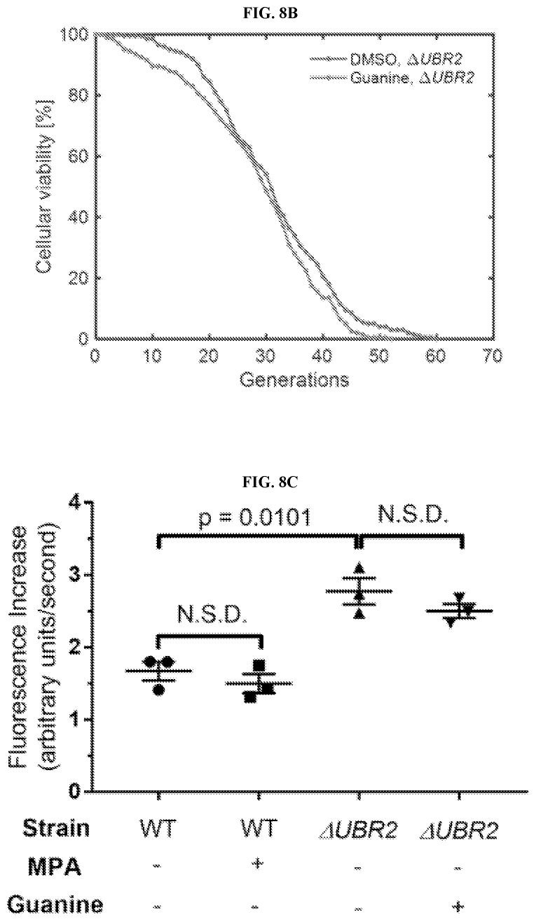

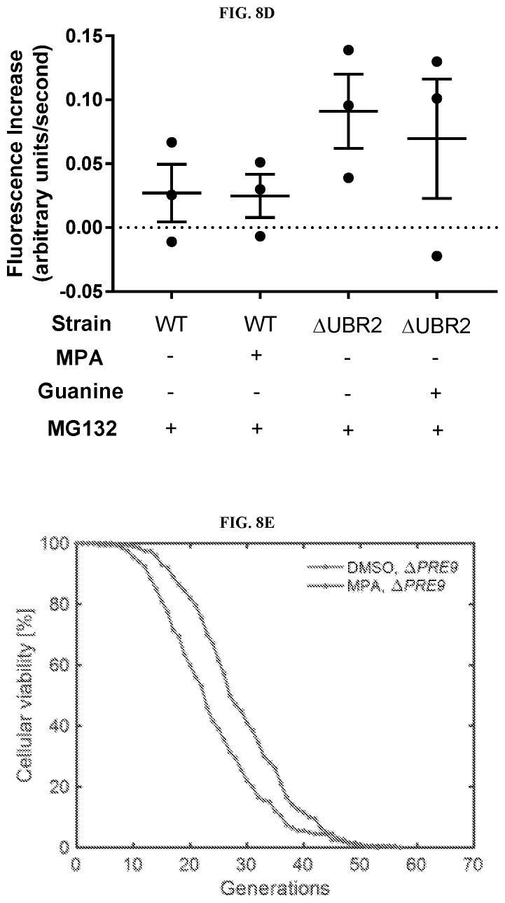

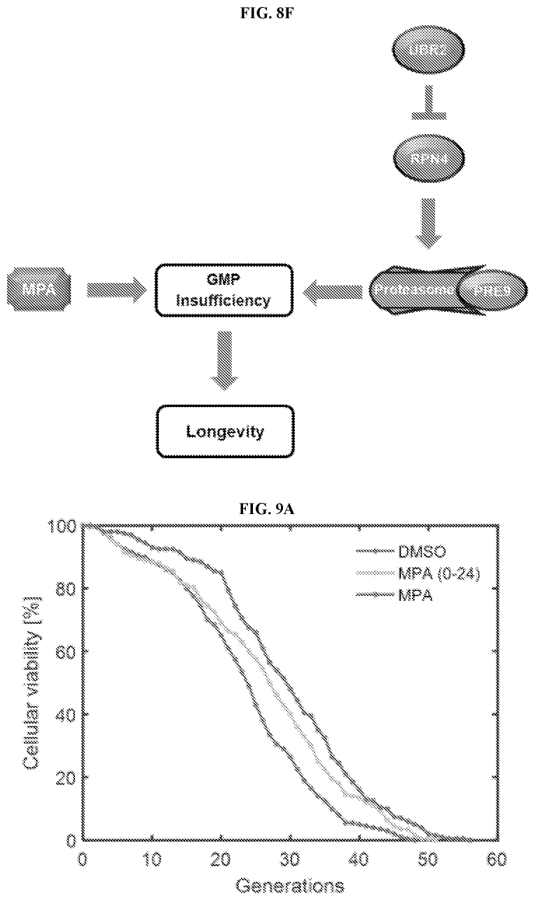

Next, it was determined whether GMP depletion extended RLS through the third major lifespan-extension pathway, the proteasome pathway. UBR2 is a ubiquitin ligase [symbol] its deletion activates the proteasome by stabilizing RPN4, a transcription factor that promotes expression of proteasome subunits (Wang, et al., J. Biol. Chem. 279, 55218-55223 (2004).). Activation of the proteasome via UBR2 deletion extends RLS independent of the nutrient sensing pathway. Therefore, UBR2 was chosen as the LPT probe gene. MPA moderately, but not significantly, extended RLS in a ΔUBR2 strain (FIG. 8A), suggesting that UBR2 deletion may activate the same lifespan extension mechanism as MPA without saturating the target. Guanine supplementation partially suppressed lifespan extension from UBR2 deletion (FIG. 8B), indicating that UBR2 deletion extends lifespan through GMP insufficiency. A role for GMP metabolism in the phenotypic effects of UBR2 deletion is reinforced by the observation that IMD proteins are among the most highly upregulated proteins in a ΔUBR2 strain. Without intending to be limited to any particular theory, these results suggest that MPA acts to extend lifespan downstream of UBR2 in the proteasome pathway.

There exist multiple steps between UBR2 deletion and proteasome activation. Therefore, it was possible that GMP depletion acted downstream of UBR2, but upstream of proteasome activation. In order to differentiate these possibilities proteasome activity was measured in wild-type cells in the presence and absence of MPA (FIGS. 8C-8D), and found that MPA did not activate the proteasome. Furthermore, guanine did not alter proteasome activity in ΔUBR2 cells (FIG. 8C). Without intending to be limited to any particular theory, this suggests that GMP regulates lifespan without modulating the proteasome. This theory was further supported by demonstrating that MPA extends RLS in a ΔPRE9 strain (FIG. 8F), in which proteasome activation did not increase RLS due to the absence of the proteasome subunit PRE9. Since deletion of UBR2 extends RLS exclusively through proteasome activation, it is reasonable to suggest that proteasome activation extends lifespan in part through depletion of GMP or its downstream metabolites (FIG. 8G).

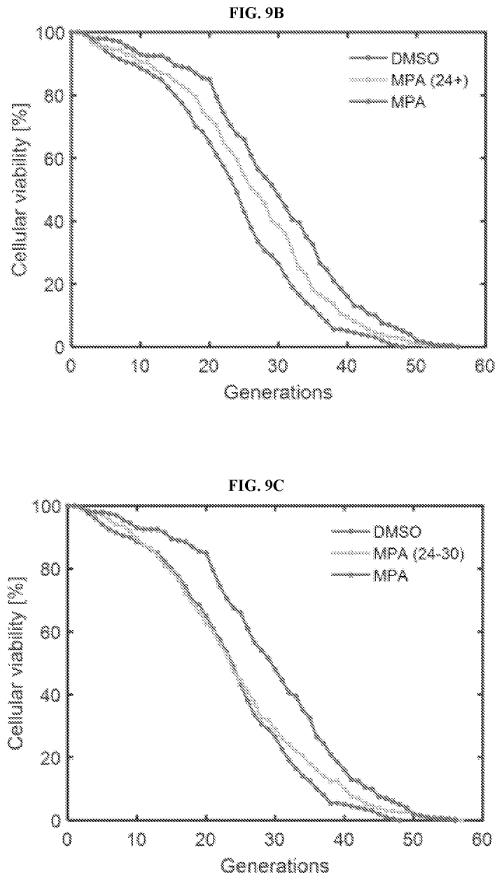

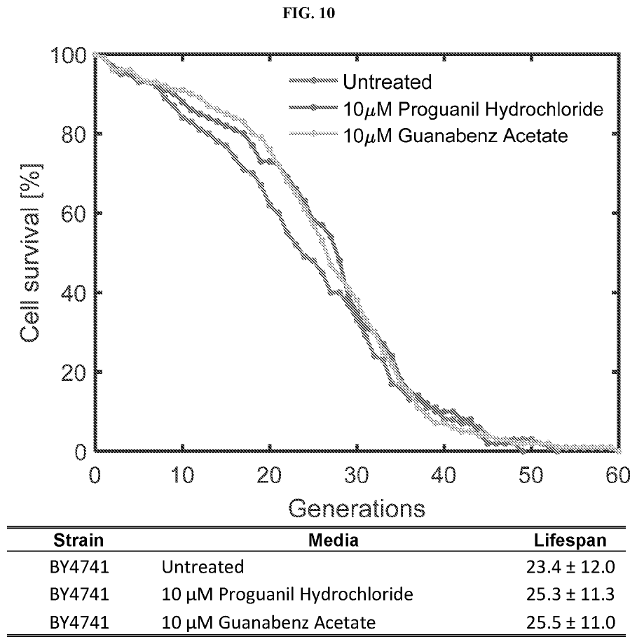

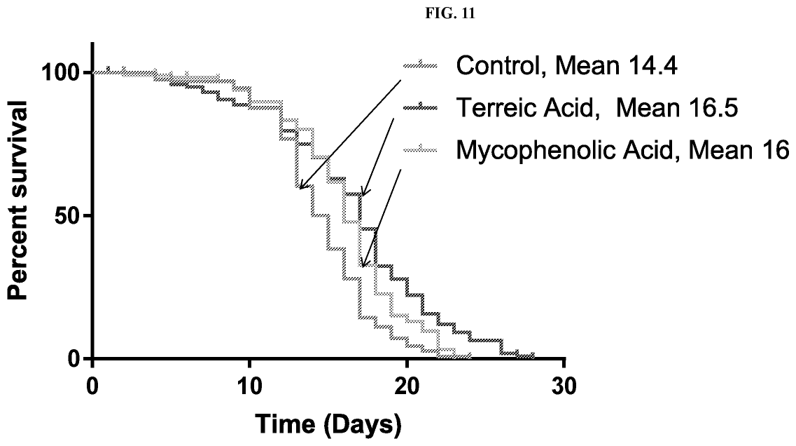

Interventions that extend lifespan may act to slow the accumulation of age-related damage, reverse age-related damage, or suppress its effects. In order to determine through which mechanism GMP insufficiency extended the lifespan, the RLS of yeast cells treated with MPA was assessed only for the first 24 hours of a Replicator experiment (FIG. 9A), only after the first 24 hours of a Replicator experiment (FIG. 9B), or with a 6-hour pulse treatment between the 24^{th} and 30^{th} hour of the experiment (FIG. 9C). It was found that MPA treatment for only part of the lifespan, either early or late, resulted in reduced lifespan extension compared to whole-lifespan treatment. Furthermore, pulse treatment resulted in little to no lifespan extension. This suggests that GMP insufficiency slows, rather than reverses, the [symbol] n of age-related damage.

### Example 5: Lifespan Extension Validation of Progruanil Hydrochloride and Guanabenz Acetate

Follow-up validation studies were further carried out for additional compounds found to demonstrate at least some lifespan extending properties in the experiments reported in Example 3. Proguanil hydrochloride and guanabenz acetate were supplied to young yeast cells throughout the duration of their lifespan. Lifespan was measured according to the procedures described elsewhere herein (see Replicator Experiments). Both guanabenz acetate and proguanil hydrochloride were found to extend the lifespan of these cells.

### Example 6: Evolutionary Conservation of Terreic Acid and Mycophenolic Acid

In order to demonstrate that the validated compounds function in an evolutionarily conserved manner, Caenorhabditis elegans (roundworms) were treated with mycophenolic acid or terreic acid for the duration of their lifespans. In each case, measurable extension of lifespan was observed (FIG. 11). These results suggest that terreic acid and mycophenolic acid act on evolutionarily-conserved targets. Without intending to be limited to any particular theory, given the evolutionary distance between C. elegans and S. cerevisiae, the observed activity raises the possibility of evolutionary conservation between the less-distantly related C. elegans and humans.

### Example 7: Testing of Additional Compounds

The High-Life method was used to screen additional compounds for lifespan extending properties. A variety of compounds were selected based on their ability to inhibit the same or related metabolic pathways as the initial positive compounds mycophenolic acid, guanabenz and progruanil hydrochloride. Certain compounds were selected for their ability to inhibit GMP production or related biological products such as adenosine monophosphate (AMP). Testing conditions were identical to those reported in Example 3. The following compounds were found to demonstrate at least some lifespan extending properties: A79922, Chlorpromazine, Quinacrine, Azathioprine, Leflunomide, Mizoribine, Methotrexate, Pemetrexed, Pentamidine, Pyrimethamine, Sulfamethoxazole, and Trimethoprim.

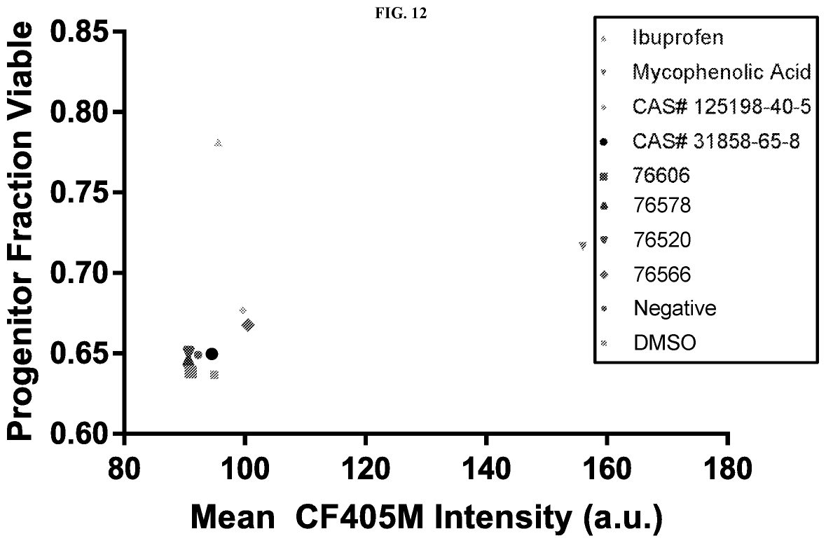

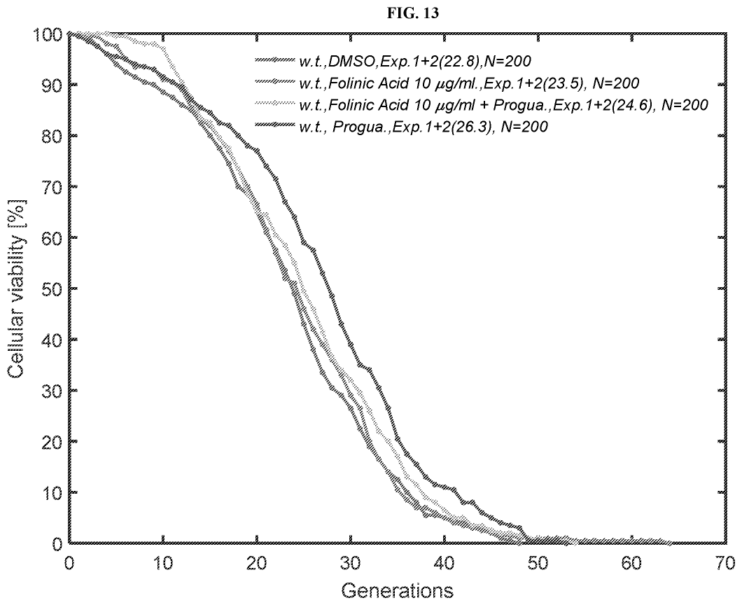

Structural analogues of mycophenolic acid were also tested for lifespan extending properties. Both (E)-6-(4-hydroxy-6-methoxy-7-methyl-3-oxo-1,3-dihydroisobenzofuran-5-yl)-4-methyl-N-(pyridin-4-ylmethyl)hex-4-enamide and (3-(2-((4-Hydroxy-6-methoxy-7 [symbol] methyl-2-oxo-1,3-dihydroisobenzofuran-5-yl)methyl)-1-methylcyclopropyl)propanoic acid) were found to demonstrate at least some lifespan extending properties (FIG. 12). Without intending to be limited to any particular theory, these results suggest that some molecular structure conserved among MPA and these structural analogues may be responsible for the lifespan extension observed.

### Example 8: Effect of Folinic Acid on Proguanil Lifespan Extension

S. cerevisiae cells in the Replicator device (see Replicator Experiments) were subjected to treatment with 10 μM proguanil, with or without 10 μg/mL folinic acid, and their replicative lifespans were measured. Proguanil is a known inhibitor of dihydrofolate reductase, an essential enzyme for the synthesis of tetrahydrofolate. When folinic acid is present, tetrahydrofolate can be synthesized via a parallel alternative pathway. The results showed that 10 μg/mL folinic acid was able to suppress the longevity effect of proguanil, suggesting that proguanil exerts its longevity effect via depletion of tetrahydrofolate.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of extending the lifespan of a subject, the method comprising administering to the subject a therapeutically effective amount of at least one compound, or a salt, solvate, enantiomer, diastereoisomer, or tautomer thereof, selected from the group consisting of:

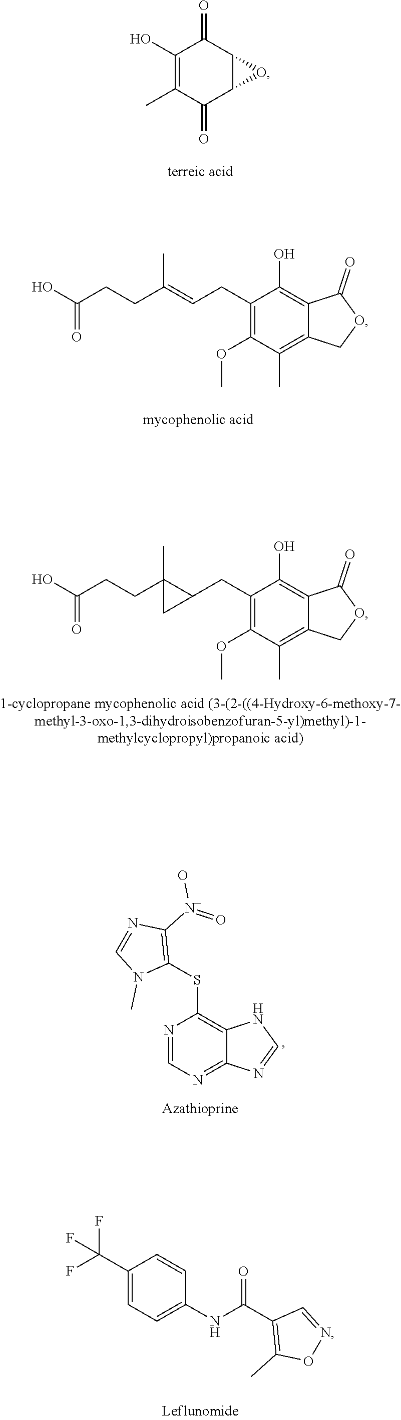

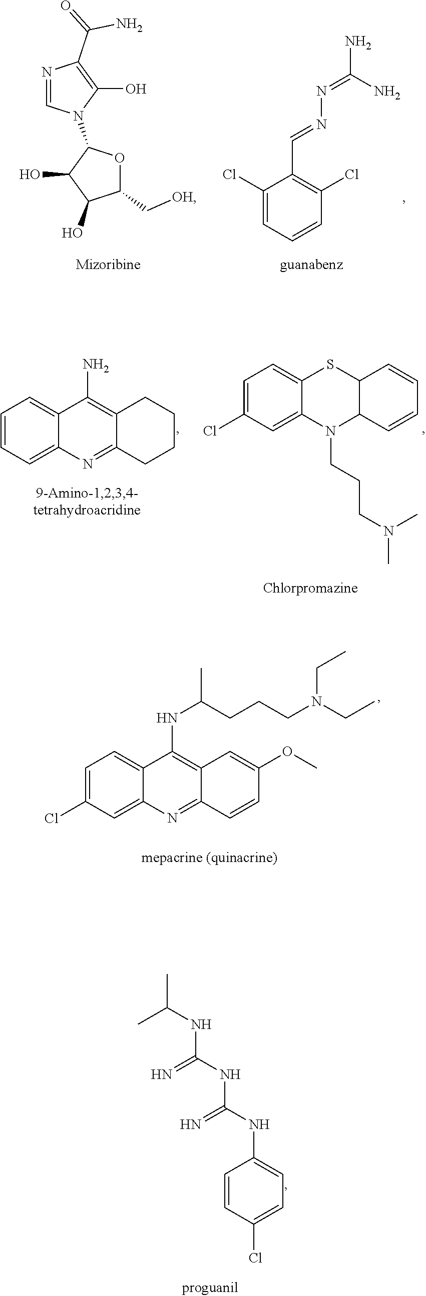

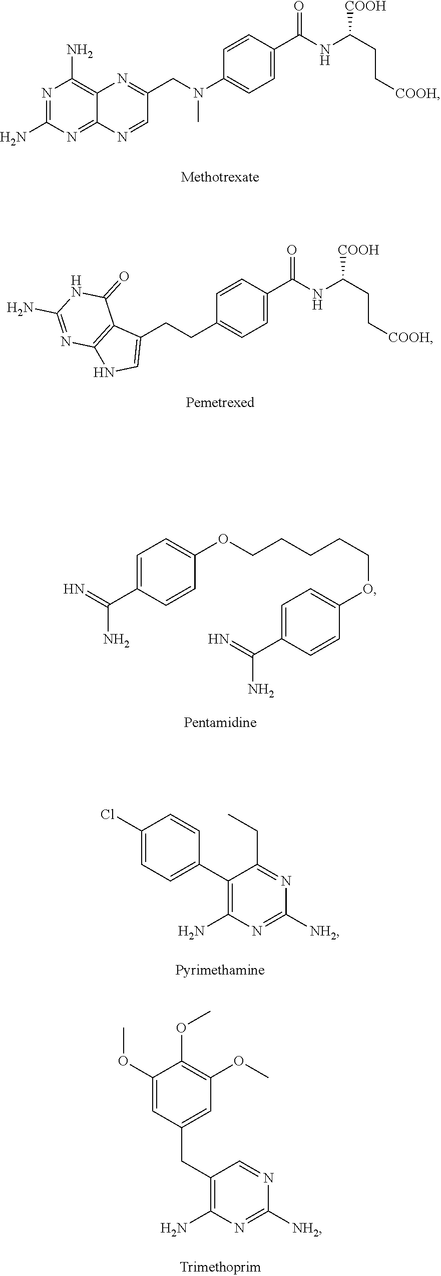 [SMILES: CN(Cc1cnc2nc(N)nc(N)c2n1)c1ccc(C(=O)N[C@@H](CCC(=O)O)C(=O)O)cc1 ; Nc1nc2[nH]cc(CCc3ccc(C(=O)N[C@@H](CCC(=O)O)C(=O)O)cc3)c2c(=O)[nH]1 ; N=C(N)c1ccc(OCCCCCOc2ccc(C(=N)N)cc2)cc1 ; CCc1nc(N)nc(N)c1-c1ccc(Cl)cc1 ; COc1cc(Cc2cnc(N)nc2N)cc(OC)c1OC]

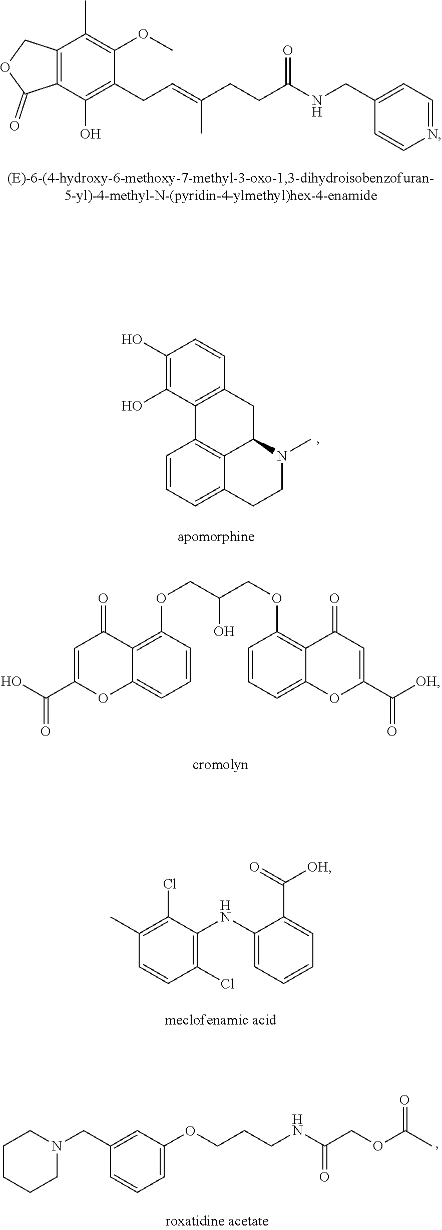 [SMILES: COc1c(C)c2c(c(O)c1C/C=C(\C)CCC(=O)NCc1ccncc1)C(=O)OC2 ; CN1CCc2cccc3c2[C@H]1Cc1ccc(O)c(O)c1-3 ; O=C(O)c1cc(=O)c2c(OCC(O)COc3cccc4oc(C(=O)O)cc(=O)c34)cccc2o1 ; Cc1ccc(Cl)c(Nc2ccccc2C(=O)O)c1Cl ; CC(=O)OCC(=O)NCCCOc1cccc(CN2CCCCC2)c1]

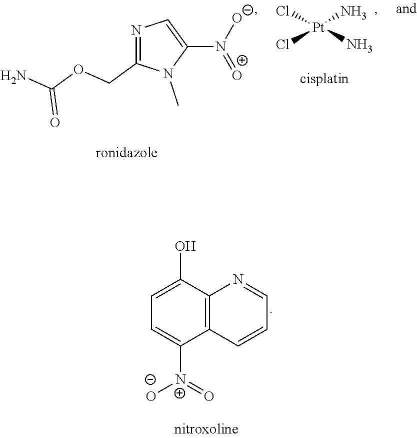

2. The method of claim 1, wherein the lifespan of the subject is extended by about 15% to about 25%.

3. The method of claim 1, wherein the lifespan of the subject is extended by about 18% to about 23%.

4. The method of claim 1, wherein the at least one compound treats an aging-related disease or disorder.

5. The method of claim 4, wherein the aging-related disease or disorder is one or more selected from the group consisting of atherosclerosis, cardiovascular disease, respiratory disease, cancer, arthritis, osteoporosis, type 2 diabetes, hypertension, Alzheimer's disease, Parkinson's disease, liver disease, kidney disease, and immunosenescence.

6. The method of claim 1, wherein the at least one compound has at least one of the following activities:
(a) alters immune response in the subject;
(b) suppresses the subject's immune system;
(c) inhibits at least one selected from the group consisting of guanosine monophosphate (GMP) synthesis, adenosine monophosphate (AMP) synthesis, and tetrahydrofolate synthesis in the subject.

7-8. (canceled)

9. The method of claim 1, wherein the at least one compound is administered as part of a pharmaceutical composition.

10. The method of claim 1, wherein the subject is further administered at least one additional agent useful for extending lifespan.

11. The method of claim 10, wherein the at least one compound and the at least one additional agent are co-formulated.

12. The method of claim 10, wherein the at least one additional agent useful for extending lifespan is selected from the group consisting of ibuprofen, rapamycin, metformin, and nicotinamide riboside.

13. The method of claim 1, wherein the subject is a eukaryotic organism.

14. The method of claim 1, wherein the subject is a mammal.

15. The method of claim 14, wherein the subject is a human.

16. A method of identifying compounds that extend the lifespan of a subject, the method comprising:
contacting “mother enriched” yeast cells with an NHS functionalized fluorophore in a growth medium, to form a first system;
contacting at least one aliquot of the first system with β-estradiol, to form a second system;
incubating the second system with a test compound or control compound, to form a third system;
contacting the third system with a WGA functionalized fluorophore and a cell viability dye, to form a fourth system; and
conducting flow cytometry on the fourth system to detect fluorescence from at least one fluorophore selected from the group consisting of the NHS functionalized fluorophore, the WGA functionalized fluorophore and the cell viability dye;
wherein the “mother enriched” yeast cells are genetically modified yeast cells wherein the replicative capacity of the “mother enriched” yeast cells is not altered while the replicative capacity of their progeny cells is restricted.

17. The method of claim 16, wherein the mean lifespan of the yeast cells is determined by conducting flow cytometry on each sample at two or more time points.

18. The method of claim 17, wherein flow cytometry is conducted at two or more time points between 0 hours and about 48 hours.

19. The method of claim 16, wherein the flow cytometry is carried out using an automated flow cytometry device.

20. The method of claim 16, wherein the at least one aliquot is part of a screening array.

21. The method of claim 20, wherein the screening array comprises a multi-well plate.

22. The method of claim 16, wherein at least one applies:
(a) the NHS functionalized fluorophore is at least one selected from the group consisting of NHS-Fluorescein and NETS-Rhodamine;
(b) the cell viability dye is propidium iodide;
(c) the WGA functionalized fluorophore is CF405M-WGA.

23-24. (canceled)
